(19) United States
(12) Patent Application Publication
(10) Pub. No.: US 2015/0050261 A1
(43) Pub. Date: Feb. 19, 2015

(54) DIETARY AND NUTRITIONAL SUPPLEMENTS CONTAINING AN OMEGA-3 FATTY ACID OF PLANT-BASED ORIGIN AND A FOLATE DERIVATIVE

(71) Applicant: Vertical Pharmaceuticals, LLC, Sayreville, NJ (US)

(72) Inventors: Chris G. Evenstad, Minnetonka, MN (US); Kelly M. Heimerl, Maple Grove, MN (US); Mike J. McBride, Maple Grove, MN (US); Ronnie Ortiz, Apple Valley, MN (US); Carrol J. Rhead, Denver, CO (US); Sami Yusuf, Blaine, MN (US); Michelle B. Zachman, Rogers, MN (US)

(21) Appl. No.: 14/461,314

(22) Filed: Aug. 15, 2014

Related U.S. Application Data
(60) Provisional application No. 61/866,840, filed on Aug. 16, 2013.

(51) Int. Cl. A61K 33/26; A61K 31/225; A61K 33/10; A61K 31/341; A61K 31/7028; A61K 31/355; A61K 31/519; A61K 31/593; A61K 31/4188; A61K 31/202; A61K 31/675

(52) U.S. Cl. CPC A61K 33/26; A61K 31/202; A61K 31/225; A61K 33/10; A61K 31/341; A61K 31/675; A61K 31/355; A61K 31/519; A61K 31/593; A61K 31/4188; A61K 31/7028
USPC 424/94.1; 424/648; 424/643; 424/638

(57) ABSTRACT

The present invention relates to dietary and nutritional supplements comprising various vitamins, minerals and nutrients, including an omega-3 fatty acid of plant-based origin and a folate derivative.

### PRIORITY CLAIM

To the fullest extent permitted by law, this non-provisional patent application claims priority to and the full benefit of the provisional patent application entitled “Pharmaceutical Compositions Containing an Omega-3 Fatty Acid of Plant-Based Origin and a Folate Derivative”, filed on Aug. 16, 2013, and assigned Ser. No. 61/866,840.

### FIELD OF THE INVENTION

The present invention relates generally to dietary and nutritional supplements, and more specifically to dietary and nutritional supplements containing an omega-3 fatty acid of plant-based origin and a folate derivative.

### SUMMARY OF THE INVENTION

The present invention provides dietary and nutritional supplements (hereinafter “supplement(s)” or “composition(s)”) comprising various vitamins, minerals and other nutrients, and methods for administering or using such compositions for dietary and nutritional supplementation in humans. In particular, the present invention provides compositions and methods designed to supplement the nutritional needs of women throughout pregnancy (i.e., the prenatal period), during the postnatal period for both lactating and non-lactating mothers, and throughout the childbearing years, and wherein such compositions and methods may be further used to improve the nutritional status of women prior to conception.

An embodiment of the present invention contemplates a supplement comprising an omega-3 fatty acid and a folate derivative.

Another embodiment of the present invention contemplates a supplement comprising an omega-3 fatty acid (preferably, docosahexaenoic acid, and more preferably, docosahexaenoic acid of plant-based origin), and a folate derivative, wherein the folate derivative is N-[4-[[[(6S)-2-amino-1,4,5,6,7,8-hexahydro-5-methyl-4-oxo-6-pteridinyl]methyl]amino]benzoyl]-L-glutamic acid, glucosamine salt (the foregoing chemical name of the referenced folate derivative is also referred to herein as either the glucosamine salt of (6S)-5-methyltetrahydrofolate, or (6S)-5-methyltetrahydrofolate glucosamine salt (and which chemical name may also be written as N-[4-[[[-2-amino-5-methyl-1,4,5,6,7,8-hexahydro-4-oxo-6(S)pteridinyl]methyl]amino]benzoyl]-L-glutamic acid, glucosamine salt)).

Another embodiment of the present invention contemplates a supplement comprising an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention contemplates a supplement comprising or consisting essentially of an omega-3 fatty acid, a folate derivative, calcium, iron, and folic acid.

Another embodiment of the present invention contemplates a supplement comprising or consisting essentially of an omega-3 fatty acid, a folate derivative, docusate, calcium, iron, and folic acid.

Another embodiment of the present invention contemplates a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium, iron, folic acid, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention contemplates a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate, calcium, iron, folic acid, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention contemplates a supplement comprising or consisting essentially of an omega-3 fatty acid, a folate derivative, calcium, ferrous fumarate, and folic acid.

Another embodiment of the present invention contemplates a supplement comprising or consisting essentially of an omega-3 fatty acid, a folate derivative, docusate, calcium, ferrous fumarate, and folic acid.

Another embodiment of the present invention contemplates a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium, ferrous fumarate, folic acid, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention contemplates a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate, calcium, ferrous fumarate, folic acid, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention contemplates a supplement comprising or consisting essentially of an omega-3 fatty acid, a folate derivative, calcium carbonate, ferrous fumarate, and folic acid.

Another embodiment of the present invention contemplates a supplement comprising or consisting essentially of an omega-3 fatty acid, a folate derivative, docusate calcium, calcium carbonate, ferrous fumarate, and folic acid.

Another embodiment of the present invention contemplates a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium carbonate, ferrous fumarate, folic acid, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention contemplates a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate calcium, calcium carbonate, ferrous fumarate, folic acid, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention contemplates a supplement comprising or consisting essentially of an omega-3 fatty acid, a folate derivative, calcium carbonate, ferrous fumarate, folic acid, and one or more of vitamin C, vitamin B6, vitamin E, vitamin D3 and/or D-biotin.

Another embodiment of the present invention contemplates a supplement comprising or consisting essentially of an omega-3 fatty acid, a folate derivative, docusate calcium, calcium carbonate, ferrous fumarate, folic acid, and one or more of vitamin C, vitamin B6, vitamin E, vitamin D3 and/or D-biotin.

Another embodiment of the present invention contemplates a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium carbonate, ferrous fumarate, folic acid, and one or more of vitamin C, vitamin B6, vitamin E, vitamin D3 and/or D-biotin, and further at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention contemplates a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate calcium, calcium carbonate, ferrous fumarate, folic acid, and one or more of vitamin C, vitamin B6, vitamin E, vitamin D3 and/or D-biotin, and further at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention contemplates a supplement comprising or consisting essentially of an omega-3 fatty acid, a folate derivative, calcium carbonate, ferrous fumarate, folic acid, vitamin C, vitamin B6, vitamin E, vitamin D3, and D-biotin.

Another embodiment of the present invention contemplates a supplement comprising or consisting essentially of an omega-3 fatty acid, a folate derivative, docusate calcium, calcium carbonate, ferrous fumarate, folic acid, vitamin C, vitamin B6, vitamin E, vitamin D3, and D-biotin.

Another embodiment of the present invention contemplates a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium carbonate, ferrous fumarate, folic acid, vitamin C, vitamin B6, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention contemplates a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate calcium, calcium carbonate, ferrous fumarate, folic acid, vitamin C, vitamin B6, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention provides a supplement comprising or consisting essentially of an omega-3 fatty acid, a folate derivative, calcium carbonate, ferrous fumarate, folic acid, iodine, vitamin C, vitamin B6, vitamin E, vitamin D3, and D-biotin.

Another embodiment of the present invention provides a supplement comprising or consisting essentially of an omega-3 fatty acid, a folate derivative, docusate calcium, calcium carbonate, ferrous fumarate, folic acid, iodine, vitamin C, vitamin B6, vitamin E, vitamin D3, and D-biotin.

Another embodiment of the present invention provides a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium carbonate, ferrous fumarate, folic acid, iodine, vitamin C, vitamin B6, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention provides a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate calcium, calcium carbonate, ferrous fumarate, folic acid, iodine, vitamin C, vitamin B6, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention provides a supplement comprising or consisting essentially of an omega-3 fatty acid, a folate derivative, calcium carbonate, ferrous fumarate, folic acid, iodine, vitamin C, vitamin B6, vitamin B12, vitamin E, vitamin D3, and D-biotin.

Another embodiment of the present invention provides a supplement comprising or consisting essentially of an omega-3 fatty acid, a folate derivative, docusate calcium, calcium carbonate, ferrous fumarate, folic acid, iodine, vitamin C, vitamin B6, vitamin B12, vitamin E, vitamin D3, and D-biotin.

Another embodiment of the present invention provides a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium carbonate, ferrous fumarate, folic acid, iodine, vitamin C, vitamin B6, vitamin B12, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention provides a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate calcium, calcium carbonate, ferrous fumarate, folic acid, iodine, vitamin C, vitamin B6, vitamin B12, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention provides a supplement comprising or consisting essentially of an omega-3 fatty acid, a folate derivative, calcium carbonate, ferrous fumarate, folic acid, iodine, vitamin C, vitamin B6, vitamin B12, choline, vitamin E, vitamin D3, and D-biotin.

Another embodiment of the present invention provides a supplement comprising or consisting essentially of an omega-3 fatty acid, a folate derivative, docusate calcium, calcium carbonate, ferrous fumarate, folic acid, iodine, vitamin C, vitamin B6, vitamin B12, choline, vitamin E, vitamin D3, and D-biotin.

Another embodiment of the present invention provides a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium carbonate, ferrous fumarate, folic acid, iodine, vitamin C, vitamin B6, vitamin B12, choline, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention provides a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate calcium, calcium carbonate, ferrous fumarate, folic acid, iodine, vitamin C, vitamin B6, vitamin B12, choline, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention provides a supplement comprising or consisting essentially of an omega-3 fatty acid, a folate derivative, calcium carbonate, ferrous fumarate, folic acid, vitamin C, vitamin B6, vitamin E, vitamin D3, D-biotin, and one or more of iodine, vitamin B12 and/or choline.

Another embodiment of the present invention provides a supplement comprising or consisting essentially of an omega-3 fatty acid, a folate derivative, docusate calcium, calcium carbonate, ferrous fumarate, folic acid, vitamin C, vitamin B6, vitamin E, vitamin D3, D-biotin, and one or more of iodine, vitamin B12 and/or choline.

Another embodiment of the present invention provides a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium carbonate, ferrous fumarate, folic acid, vitamin C, vitamin B6, vitamin E, vitamin D3, D-biotin, and one or more of iodine, vitamin B12 and/or choline, and further at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention provides a supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate calcium, calcium carbonate, ferrous fumarate, folic acid, vitamin C, vitamin B6, vitamin E, vitamin D3, D-biotin, and one or more of iodine, vitamin B 12 and/or choline, and further at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention provides a supplement comprising or consisting essentially of an omega-3 fatty acid, a folate derivative, and one or more of the following ingredients: calcium carbonate; ferrous fumarate; folic acid; vitamin A; vitamin C; vitamin D; vitamin E, vitamin B1; vitamin B2; vitamin B3; vitamin B5; vitamin B6; vitamin B12; choline; vitamin D3; D-biotin; iodine; iron; zinc; copper; calcium; magnesium; an omega-3 fatty acid; and/or, any and all pharmaceutically acceptable forms of the foregoing.

Another embodiment of the present invention provides a supplement comprising or consisting essentially of an omega-3 fatty acid, a folate derivative, and one or more of the following ingredients: docusate calcium; calcium carbonate; ferrous fumarate; folic acid; vitamin A; vitamin C; vitamin D; vitamin E, vitamin B1; vitamin B2; vitamin B3; vitamin B5; vitamin B6; vitamin B12; choline; vitamin D3; D-biotin; iodine; iron; zinc; copper; calcium; magnesium; an omega-3 fatty acid; and/or, any and all pharmaceutically acceptable forms of the foregoing.

Another embodiment of the present invention provides a supplement comprising or consisting essentially of (i) an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), and one or more of the following ingredients: calcium carbonate; ferrous fumarate; folic acid; vitamin A; vitamin C; vitamin D; vitamin E, vitamin B1; vitamin B2; vitamin B3; vitamin B5; vitamin B6; vitamin B12; choline; vitamin D3; D-biotin; iodine; iron; zinc; copper; calcium; magnesium; an omega-3 fatty acid; and/or, any and all pharmaceutically acceptable forms of the foregoing; and, (ii) at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Another embodiment of the present invention provides a supplement comprising or consisting essentially of (i) an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), and one or more of the following ingredients: docusate calcium; calcium carbonate; ferrous fumarate; folic acid; vitamin A; vitamin C; vitamin D; vitamin E, vitamin B1; vitamin B2; vitamin B3; vitamin B5; vitamin B6; vitamin B12; choline; vitamin D3; D-biotin; iodine; iron; zinc; copper; calcium; magnesium; an omega-3 fatty acid; and/or, any and all pharmaceutically acceptable forms of the foregoing; and, (ii) at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

Dosage forms of each of the several embodiments of the supplements described herein, including the foregoing embodiments, may be formulated for oral administration and, to that end, may be delivered as, provided as or otherwise contained in one or more swallowable, chewable or dissolvable (as applicable) tablets, caplets, pills, capsules, softgels, and, more preferably, in a swallowable, single softgel capsule. The various supplements described herein may be administered to a human female before pregnancy (e.g., prior to conception), during pregnancy, after pregnancy, to lactating and non-lactating mothers, and throughout the childbearing years, or a combination thereof. Moreover, the various supplements described herein may be administered once daily or as directed by a physician.

These and other features and advantages of the compositions and methods of the present invention will become apparent to those of ordinary skill in the art after reading the following Detailed Description of the Invention.

### DETAILED DESCRIPTION OF THE INVENTION

For simplicity and illustrative purposes, the principles of the present invention are described by referring to various exemplary embodiments thereof. It is understood that the present invention is not limited to the particular examples, methodologies, protocols, fillers, and excipients, etc., described herein, as these may vary. It is also to be understood that the terminology used herein is used for the purpose of describing particular embodiments only, and is not intended to limit the scope of the present invention. Moreover, although certain methods may be described with reference to certain steps that are presented herein in a certain order, in many instances, these steps may be performed in any order as would be appreciated by one of ordinary skill in the art, and thus the methods are not limited to the particular arrangement of steps disclosed herein. It must be noted that as used herein and in the appended claims, the singular forms “a,” “an,” and “the” include the plural reference unless the context clearly dictates otherwise. Thus, for example, a reference to “a vitamin” is a reference to one or more vitamins and includes equivalents thereof known to those skilled in the art and so forth.

Unless defined otherwise, all technical and scientific terms used herein have the same meanings as commonly understood by one of ordinary skill in the art to which this invention belongs. Specific methods and materials are described, although any methods and materials similar or equivalent to those described herein can be used in the practice or testing of the present invention.

The phrase “pharmaceutically acceptable,” as used herein, refers to those compounds, materials, compositions and/or dosage forms which are, within the scope of sound pharmaceutical/medical judgment, suitable for use in contact with the tissues of human beings and animals without excessive toxicity, irritation, allergic response, or other problem or complication, commensurate with a reasonable benefit/risk ratio. Thus, the phrase “pharmaceutically acceptable excipient” (or the plural form thereof), as used herein, refers to such suitable compounds and materials defined above that may be added to the dosage form to assist in satisfactory processing of the dosage form or provide desirable physical characteristics to the dosage form, and may include, for example but without limitation, carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

The term “administration” or “administered” or derivatives thereof defines a composition that is able to be given to a human patient. Likewise, “administering” refers to the act of giving a composition to a patient or otherwise making such composition available to a patient or the patient taking a composition.

As used herein, the term “about,” when located before a dosage amount or dosage range of a specific ingredient, refers to an amount or range closely above and/or closely below the stated amount or range that does not manifestly alter the therapeutic effect of the specific ingredient from the stated amount or range and is meant to encompass at least all equivalents of that amount.

### Ingredients, Dosage Forms, and Administration of the Supplements

Dosage forms of each of the several embodiments of the supplements described herein are preferably formulated for oral administration and, to that end, may be delivered as, provided as or otherwise contained in one or more swallowable, chewable or dissolvable (as applicable) tablets, caplets, pills, capsules, softgels, and, more preferably, in a swallowable, single softgel capsule. To formulate and prepare the various embodiments of the supplements of the present invention, the ingredients identified and described hereinbelow may be combined in intimate admixture according to conventional compounding techniques, to thereby provide any of the supplements hereof in any selected dosage form.

Docosahexaenoic Acid (e.g., as DHA, 53% in Sunflower Oil). Docosahexaenoic Acid or DHA is an omega-3 fatty acid that functions as a major structural fat in the brain, where it can account for up to 97% of the omega-3 fats, and in the retina, where it can account for up to 93% of the omega-3 fats. The DHA product used in the present invention is plant-based (i.e., derived from microalgae), rather than fish oil -based. Although fish oil -based DHA may be used if and as desired, the use of plant-based DHA provides a DHA product that is essentially free of, substantially excludes, eliminates, or otherwise reduces the presence of, eicosapentaenoic acid (EPA), which may be present in fish oil (and thus in fish oil -based DHA), and which may disrupt the EPA:DHA balance in the developing baby and potentially cause fetal developmental problems in pregnant women. As such, the DHA used in the present invention is prepared or derived from Crypthecodinium cohnii dinoflagellate microalgae (and/or alternatively from Schizocytrium microalgae, Thraustochytrium microalgae, and/or mixtures of Crypthecodinium cohnii, Schizocytrium and Thraustochytrium microalgae). Such plant-based DHA products, and the processes for the preparation and manufacture of same, are described in U.S. Pat. Nos. 5,407,957, 5,492,938 and 6,977,167, the disclosures of which are incorporated herein by reference in their entirety. The raw, plant-based DHA material used in the present invention is available from DSM Nutritional Products, Inc. (Parsippany, N.J.) under the trademark life'sDHA™, and is available as a dispersion in sunflower oil with small amounts of antioxidants (ascorbyl palmitate and alpha-tocopherols). For the present invention, the 53% DHA dispersion may be used (i.e., a “DHA, 53% in sunflower oil” blend). Accordingly, and for example, about 660 mg of such a “DHA, 53% in sunflower oil” blend would provide about 350 mg of docosahexaenoic acid (i.e., about 660 mg×53%=about 350 mg), and about 1246 mg of such a “DHA, 53% in sunflower oil” blend would provide about 660.4 mg of docosahexaenoic acid (i.e., about 1246 mg×53%=about 660.4 mg)—such calculations being known to those of ordinary skill in the art. For clarity, the specific amount or range of specific amounts of docosahexaenoic acid referenced in any of the general or specific examples of the supplements described herein refers to the amount of docosahexaenoic acid contained in and provided by the specific supplements so described, and does not refer to the amount of “DHA, 53% in sunflower oil” blend required to provide the specific amount or range of specific amounts of docosahexaenoic acid referenced in any specific examples in which it is indicated that docosahexaenoic acid may be provided in the form of a “DHA, 53% in sunflower oil” blend.

Calcium (e.g., as Calcium carbonate 98.0%, USP). Calcium is an important mineral for the body as it is the major component of bones and teeth. It is also a critical component in muscle contraction, blood vessel expansion and contraction, secretion of hormones or enzymes in the body, and impulse transmission about the nervous system. In the present invention, calcium carbonate (98.0%, USP) may be used, and may be supplied as a dry powder. As an alternative to calcium carbonate, or in addition thereto, tribasic calcium phosphate, NF, may be used.

Folic Acid (e.g., as Folic Acid, 97%, USP/FCC). Folic acid is the synthetic form of folate, a water-soluble B-complex vitamin (B9) that aids in cytogenesis. Because pregnancy involves rapid cytogenesis and cell growth, folic acid is an important prenatal dietary supplement. In the present invention, folic acid (97%, USP/FCC) may be supplied as a dry powder.

Folate Derivative. The folate derivative used in the present invention is N-[4-[[[(6S)-2-amino-1,4,5,6,7,8-hexahydro-5-methyl-4-oxo-6-pteridinyl]methyl]amino]benzoyl]-L-glutamic acid, glucosamine salt (the foregoing chemical name of the referenced folate derivative is also referred to herein as either the glucosamine salt of (6S)-5-methyltetrahydrofolate, or (6S)-5-methyltetrahydrofolate glucosamine salt (and which chemical name may also be written as N-[4-[[[-2-amino-5-methyl-1,4,5,6,7,8-hexahydro-4-oxo-6(S)pteridinyl]methyl]amino]benzoyl]-L-glutamic acid, glucosamine salt)), and has the following structural formula:

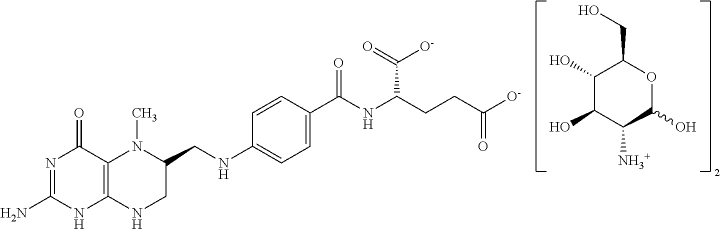

In the present invention, (6S)-5-methyltetrahydrofolate glucosamine salt may be supplied as a dry powder, and is available from Gnosis P.S.A. (Milan, Italy (or Gnosis USA, Inc. (Doylestown, Pa.)) under the trademark Quatrefolic®, and assigned CAS Number 1181972-37-1. The foregoing specific folate derivative, and alternate folate derivatives, are described in U.S. Pat. No. 7,947,662, the disclosure of which is incorporated herein by reference in its entirety.

Iron (e.g., as Ferrous Fumarate 97.0%, USP). Iron is an essential mineral in the body. In addition to cellular growth, iron is utilized during mitosis and meiosis, and during oxygen transport via the heme group. Iron is also a key component in oxidative phosphorylation during ATP production in the body. In the present invention, ferrous fumarate (97%, USP) may be used, and may be supplied as a dry powder.

Docusate (e.g., as Docusate Calcium 50% in Corn Oil, USP/NF). Docusate is a stool softener that may be added to any of the formulations to abrogate the constipating effects of iron. In the present invention, docusate calcium may be used, and may be supplied as an oil suspension (e.g., docusate calcium, 50% in Corn Oil, USP/NF). As an alternative to docusate calcium, or in addition thereto, docusate sodium may be used.

Vitamin C (e.g., as Ascorbic acid, 99%, USP/FCC). Vitamin C (or ascorbic acid) is an important physiological antioxidant; plays a role in protein metabolism; supplements the function of the immune system; and is a requisite component in biosynthesis of collagen, L-carnitine and neurotransmitters. In the present invention, Vitamin C (99%, USP/FCC) may be supplied as a dry powder.

Vitamin B6 (e.g., as Pyridoxine HCl, 99%, USP/FCC). Vitamin B6 functions in conjunction with a myriad enzymes as part of protein metabolism; plays a role in red blood cell metabolism; and supports the function of the immune and nervous systems. In the present invention, Vitamin B6 (pyridoxine HCl, 99%, USP/FCC) may be supplied as a dry powder.

Vitamin E (e.g., as d-alpha-tocopherol, 67.1% in Soybean Oil). Vitamin E is a fat-soluble antioxidant involved in immune system functions, cell signaling, regulation of gene expression, and other metabolic processes. In the present invention, Vitamin E (as d-alpha-tocopherol, 67.1% in soybean oil, and standardized at 1000 IU/gram) may be supplied as a transparent, amber-colored, viscous oil suspension, and is available from BASF Corporation (North America) under the trademark Covitol® (F1000-2). (E.g., reference herein to about 20.13 mg of Vitamin E (as d-alpha-tocopherol) equates to about 30 IU of Vitamin E (as d-alpha-tocopherol)).

Vitamin D3 (e.g., as Cholecalciferol, 2.5% in Corn Oil, USP). Vitamin D3 is a fat-soluble vitamin that promotes calcium uptake in the gut; helps maintain homeostatic serum concentrations of calcium and phosphate; and, is crucial for bone growth and bone restructuring. In the present invention, Vitamin D3 (cholecalciferol) may be supplied as a 2.5% dispersion in corn oil (USP). (E.g., reference herein to about 20.625 mcg of Vitamin D3 (as cholecalciferol) equates to about 825 IU of Vitamin D3 (as cholecalciferol)).

Biotin (e.g., as D-Biotin 98.5%, USP). Biotin is a water-soluble B-complex vitamin (B7) serving as a coenzyme in biosynthesis. Biotin is necessary for cell growth, fatty-acid production, metabolism, and gluconeogenesis. In the present invention, D-biotin (98.5%, USP) may be used and may be supplied as a dry powder.

Pharmaceutically Acceptable Excipients. Pharmaceutically acceptable excipients may include, for example but without limitation, carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. Pharmaceutically acceptable excipients are well known to those of ordinary skill in the art.

For example, carriers, binders or fillers for use in the present invention may include, by way of example and without limitation, acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, polyethylene glycol, wax, beeswax, and others known to those of ordinary skill in the art.

Disintegrants (including permeabilising and wicking agents) for use in the present invention may include, by way of example and without limitation, starches, such as corn starch, potato starch, pre-gelatinized and modified starches thereof, cellulosic agents, such as Ac-di-sol, montrnorrilonite clays, cross-linked PVP, sweeteners, bentonite, microcrystalline cellulose, croscarmellose sodium, alginates, sodium starch glycolate, gums, such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents, such as hydroxymethylcellulose, hydroxypropylcellulose and hydroxyopropylmethylcellulose.

Diluents for use in the present invention may include, by way of example and without limitation, microcrystalline cellulose, sucrose, dicalcium phosphate, starches, lactose and polyols of less than 13 carbon atoms, such as mannitol, xylitol, sorbitol, maltitol and pharmaceutically acceptable amino acids, such as glycin, and their mixtures.

Lubricants for use in the present invention may include, by way of example and without limitation, stearic acid, calcium stearate, magnesium stearate, zinc stearate, talc, mineral and vegetable oils, benzoic acid, poly(ethylene glycol), glyceryl behenate, stearyl futmarate, and others known to those of ordinary skill in the art. Glidants for use in the present invention may include, by way of example and without limitation, silicon dioxide, colloidal or fumed silica, magnesium stearate, calcium stearate, stearic acid, cornstarch, talc and others known to those of ordinary skill in the art. Viscocity agents and emulsifying agents may include, by way of example and without limitation, lecithin (e.g., soy lecithin 62% in soybean oil, NF).

Coatings (including polymeric films) for use in the present invention may include, by way of example and without limitation, vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, glycerin, and natural gums and resins such as zein, gelatin, shellac and acacia. Flavorants for use in the present invention may include, by way of example and without limitation, ethyl vanillin.

Colorants for use in the present invention may include, by way of example and without limitation, FD&C Red No. 3, FD&C Red No. 20, FD&C Red No. 40, FD&C Yellow No. 6, FD&C, FD&C Blue No. 1, FD&C Blue No. 2, FD&C Green No. 5, FD&C Orange No. 5, D&C Red No. 8, caramel, pigments, dyes, tints, titanium dioxide (e.g., an opacifier), natural coloring agents, such as grape skin extract, beet red powder, beta carotene, annato, carmine, turneric, paprika and others known to those of ordinary skill in the art. It is recognized that no colorant is required in the compositions described herein. Pharmaceutical inks for use in the present invention may include, by way of example and without limitation, white opacode, which contains ammonium hydroxide, isopropyl alcohol, n-butyl alcohol, propylene glycol, shellac glaze in SD-45 alcohol, simethicone, and titanium dioxide.

The compositions and methods of the present invention, when, for example, in the dosage form of a capsule, may include at least the following specific pharmaceutically acceptable excipients: lecithin (e.g., ADM™ Capsulec™ 62 UB Lecithin, Product No. 700315 (Archer Daniels Midland Company (Decature, Ill.)), used in the formulation to adjust the flow of the capsule fill ingredients and to aid in the dissolution/suspension of hydrophilic ingredients; wax (e.g., Yellow Beeswax Pastilles, N.F., Strahl & Pitsch Code: SP-420P (Strahl & Pitsch Inc. (W. Babylon, N.Y.)), used in the formulation as a thickening agent to exert control over the flow and viscosity of the capsule fill ingredients; and, soybean oil, used in the formulation as a solvent and/or as a viscosity or emulsifying agent for various ingredients (e.g.: soy lecithin, 62% in Soybean Oil, NF; and, Vitamin E (d-alpha-tocopherol), 67.1% in Soybean Oil).

The compositions and methods of the present invention may include or use any combination (or alternate pharmaceutically acceptable forms) of the vitamins, minerals, nutrients, and other ingredients described above. Moreover, the various compositions and methods of the present invention may include or use any one or more additional or alternate vitamins, minerals and/or nutrients, including, without limitation, vitamin D, iodine (e.g., as potassium iodide, 99%, USP), vitamin B1, vitamin B2, vitamin B3, vitamin B5, vitamin B12 (e.g., as cyanocobalamin, 1% in Gelatin, USP), choline, vitamin A, vitamin E, iron, zinc, copper, calcium, magnesium, an omega-3 fatty acid, and any and all pharmaceutically acceptable forms of the foregoing, and any one or more of the pharmaceutically acceptable excipients described above. Furthermore, the vitamins, minerals, nutrients and other ingredients may be included or used in any specific supplied form as described above, or may be provided in any alternate form conducive to any preferred or selected manufacturing process.

In any of the specific embodiments described herein, the vitamins, minerals, nutrients and/or other ingredients of the present invention, may be included in overages. Adding overages of these compounds may be necessary to meet the amounts claimed on the product label and product insert to ensure that those recited amounts are met throughout the shelf life of the product. Indeed, because of US regulatory requirements that label values reflect minimum contents of these vitamins, minerals, nutrients and/or other ingredients, deviations in actual content from label values are usually thought to tend toward overages. In a specific embodiment, one or more of the vitamins, minerals, nutrients and/or other ingredients may be included in the compositions and methods of the present invention in overages of the recited, specific label amounts of about 0% to about 150% of the label amount, although the overages are dependant on the stability of each ingredient. Overages may be larger for some vitamins, particularly those that are less stable and more likely to deteriorate with a long shelf life, those that have other functions (such as antioxidants) in the product itself; for minerals, excess amounts with large overages are probably less likely because of their increased bulk and shelf life stability. Accordingly, when overages are included for any specific vitamins, minerals, nutrients and/or other ingredients, at some point in time, those ingredients with overages will degrade so that they fall within the amounts provided in the specific label. Thus, there is no literal difference between the amounts for any vitamins, minerals, nutrients and/or other ingredients that include overages, and those amounts listed on the specific label. Furthermore, overages provide an equivalent efficacy of the ingredient over the shelf life of the product. Accordingly, any vitamins, minerals, nutrients and/or other ingredients provided in overage amounts is an insubstantial change and performs substantially the same function, in substantially the same way, and leads to substantially the same result as that same ingredient in the amounts as provided on the specific label.

The supplements of the present invention may include multiple vitamins, minerals, nutrients and/or other ingredients in a single dosage form (e.g., a single, swallowable (or dissolvable or chewable, as appropriate) tablet, caplet, pill, capsule, or softgel). A single dosage form may facilitate or improve patient compliance, particularly in those patients who may experience difficulties in taking multiple pills. Alternatively, the supplements of the present invention may include single or multiple vitamins, minerals, nutrients and/or other ingredients in more than one dosage form. By way of non-limiting example, any omega-3 fatty acids (e.g., DHA) may be included in one dosage form, whereas the other vitamins, minerals, nutrients and remaining ingredients may be included in one or more separate dosage forms. Such multiple and separate dosage forms may be packaged in any convenient manner to facilitate co-administration of the multiple dosage forms to deliver an appropriate therapeutic amount of the supplements. In addition, and as desired, dosage forms of the supplements of the present invention (whether swallowable, dissolvable or chewable) may be individually wrapped, packaged as multiple units on paper strips, blister packed, or packaged in bottles or vials of any size, without limitation.

In a specific embodiment, a dosage form of any of the supplements of the present invention is formulated as a swallowable, single softgel capsule. In general, capsules are solid dosage forms in which therapeutic agents are enclosed in a soluble gelatin wall. The wall can be made of either soft or hard gelatin. Soft gelatin capsules (“softgels” or “softgel capsules”) comprise a soft, globular, gelatin wall. The gelatin may be plasticized by the addition of additives such as glycerin, sorbitol, or a similar types of polyols. Softgel capsules offer a convenient dosage form for the administration of vitamins, minerals, dietary supplements, pharmaceuticals, and the like, and may provide advantages over more traditional dosage forms such as tablets, hard-shell capsules, and liquids. These advantages may include patient compliance and consumer preference, improved bioavailability, enhanced drug stability due to less exposure of ingredients sensitive to oxygen, excellent dose uniformity, and product differentiation. Commercially available softgel capsules come in a wide variety of sizes and shapes including, for example, round, oval, oblong, or tubular. These softgel capsules are formed by joining two identical halves to form a shell encapsulating a fill. Upon ingestion by the consumer (or on contact with water), moisture causes the capsule to come apart at the seams where the two halves are joined, thereby releasing the fill or contents of the capsule.

Methods for the preparation of softgel capsules are known in the art. One method is known as the plate process, in which a set of molds is used. A warm sheet of gelatin is laid over a lower plate and the liquid fill is poured thereon, whereupon a second sheet of gelatin is then placed thereover, followed by the top plate. The set is then placed under pressure to form the desired capsule. The sheets of gelatin used to form the softgel capsules may be colored or uncolored.

The second method is known as the rotary-die process. In the rotary-die process, soft gelatin capsules are made by continuously casting two separate ribbons of molten or flowable gelatin into two separate rotating dies of an encapsulation machine to produce soft, elastic gelatin capsules. Dyes or other suitable coloring material can be added to the molten gelatin to produce soft gelatin capsules of various colors. In addition, if different dyes are added to the different molten gelatin masses, then softgels with halves of different colors can be made. A more detailed description of the well known soft gelatin capsule art may be found in Ebert, “Soft Elastic Gelatin Capsules: A Unique Dosage Form,” Pharmaceutical Technology, October 1977 and in “The Theory and Practice of Industrial Pharmacy”, Chapter 13, Lachman et al., published by Lea and Febiger, 1970. The rotary die process described in the foregoing references are incorporated herein by reference as being descriptive of the rotary die process that may be utilized in preparing softgel dosage forms of the various supplements of the present invention.

The various supplements of the present invention, including the supplements set forth in the examples of the general and specific embodiments of the present invention described herein, may be administered to a human female before pregnancy (e.g., prior to conception), during pregnancy, after pregnancy, to lactating and non-lactating mothers, and throughout the childbearing years, or a combination thereof. In particular, as a prescription prenatal/postnatal supplement, the supplements hereof may be indicated to provide vitamin/mineral and plant-based DHA supplementation throughout pregnancy, during the postnatal period for both lactating and non-lactating mothers, and throughout the childbearing years, and may be useful in improving the nutritional status of women prior to conception. Moreover, the various supplements described herein, including any prescription prenatal/postnatal supplement based thereon, may be administered once daily or as directed by a physician, and may be provided in the dosage form of a swallowable, single softgel capsule.

### Examples of General Embodiments of the Present Invention

Examples of general embodiments of the present invention include, but are not limited to, the following:

(1) A supplement comprising an omega-3 fatty acid and a folate derivative.

(2) A supplement comprising an omega-3 fatty acid (preferably, docosahexaenoic acid, and more preferably, docosahexaenoic acid of plant-based origin), and a folate derivative, wherein the folate derivative is N-[4-[[[(6S)-2-amino-1,4,5,6,7,8-hexahydro-5-methyl-4-oxo-6-pteridinyl]methyl]amino]benzoyl]-L-glutamic acid, glucosamine salt (the foregoing chemical name of the referenced folate derivative is also referred to herein as either the glucosamine salt of (6S)-5-methyltetrahydrofolate, or (6S)-5-methyltetrahydrofolate glucosamine salt (and which chemical name may also be written as N-[4-[[[-2-amino-5-methyl-1,4,5,6,7,8-hexahydro-4-oxo-6(S) pteridinyl]methyl]amino]benzoyl]-L-glutamic acid, glucosamine salt)).

(3) A supplement comprising an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(4) A supplement comprising or consisting essentially of an omega-3 fatty acid, a folate derivative, calcium, iron, and folic acid.

(5) A supplement comprising or consisting essentially of an omega-3 fatty acid, a folate derivative, docusate, calcium, iron, and folic acid.

(6) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium, iron, folic acid, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(7) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate, calcium, iron, folic acid, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(8) A supplement comprising or consisting essentially of an omega-3 fatty acid, a folate derivative, calcium, ferrous fumarate, and folic acid.

(9) A supplement comprising or consisting essentially of an omega-3 fatty acid, a folate derivative, docusate, calcium, ferrous fumarate, and folic acid.

(10) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium, ferrous fumarate, folic acid, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(11) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate, calcium, ferrous fumarate, folic acid, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(12) A supplement comprising or consisting essentially of an omega-3 fatty acid, a folate derivative, calcium carbonate, ferrous fumarate, and folic acid.

(13) A supplement comprising or consisting essentially of an omega-3 fatty acid, a folate derivative, docusate calcium, calcium carbonate, ferrous fumarate, and folic acid.

(14) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium carbonate, ferrous fumarate, folic acid, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(15) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate calcium, calcium carbonate, ferrous fumarate, folic acid, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(16) A supplement comprising or consisting essentially of an omega-3 fatty acid, a folate derivative, calcium carbonate, ferrous fumarate, folic acid, and one or more of vitamin C, vitamin B6, vitamin E, vitamin D3 and/or D-biotin.

(17) A supplement comprising or consisting essentially of an omega-3 fatty acid, a folate derivative, docusate calcium, calcium carbonate, ferrous fumarate, folic acid, and one or more of vitamin C, vitamin B6, vitamin E, vitamin D3 and/or D-biotin.

(18) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium carbonate, ferrous fumarate, folic acid, and one or more of vitamin C, vitamin B6, vitamin E, vitamin D3 and/or D-biotin, and further at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(19) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate calcium, calcium carbonate, ferrous fumarate, folic acid, and one or more of vitamin C, vitamin B6, vitamin E, vitamin D3 and/or D-biotin, and further at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(20) A supplement comprising or consisting essentially of an omega-3 fatty acid, a folate derivative, calcium carbonate, ferrous fumarate, folic acid, vitamin C, vitamin B6, vitamin E, vitamin D3, and D-biotin.

(21) A supplement comprising or consisting essentially of an omega-3 fatty acid, a folate derivative, docusate calcium, calcium carbonate, ferrous fumarate, folic acid, vitamin C, vitamin B6, vitamin E, vitamin D3, and D-biotin.

(22) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium carbonate, ferrous fumarate, folic acid, vitamin C, vitamin B6, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(23) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate calcium, calcium carbonate, ferrous fumarate, folic acid, vitamin C, vitamin B6, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(24) A supplement comprising or consisting essentially of an omega-3 fatty acid, a folate derivative, calcium carbonate, ferrous fumarate, folic acid, iodine, vitamin C, vitamin B6, vitamin E, vitamin D3, and D-biotin.

(25) A supplement comprising or consisting essentially of an omega-3 fatty acid, a folate derivative, docusate calcium, calcium carbonate, ferrous fumarate, folic acid, iodine, vitamin C, vitamin B6, vitamin E, vitamin D3, and D-biotin.

(26) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium carbonate, ferrous fumarate, folic acid, iodine, vitamin C, vitamin B6, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(27) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate calcium, calcium carbonate, ferrous fumarate, folic acid, iodine, vitamin C, vitamin B6, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(28) A supplement comprising or consisting essentially of an omega-3 fatty acid, a folate derivative, calcium carbonate, ferrous fumarate, folic acid, iodine, vitamin C, vitamin B6, vitamin B12, vitamin E, vitamin D3, and D-biotin.

(29) A supplement comprising or consisting essentially of an omega-3 fatty acid, a folate derivative, docusate calcium, calcium carbonate, ferrous fumarate, folic acid, iodine, vitamin C, vitamin B6, vitamin B12, vitamin E, vitamin D3, and D-biotin.

(30) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium carbonate, ferrous fumarate, folic acid, iodine, vitamin C, vitamin B6, vitamin B12, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(31) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate calcium, calcium carbonate, ferrous fumarate, folic acid, iodine, vitamin C, vitamin B6, vitamin B12, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(32) A supplement comprising or consisting essentially of an omega-3 fatty acid, a folate derivative, calcium carbonate, ferrous fumarate, folic acid, iodine, vitamin C, vitamin B6, vitamin B12, choline, vitamin E, vitamin D3, and D-biotin.

(33) A supplement comprising or consisting essentially of an omega-3 fatty acid, a folate derivative, docusate calcium, calcium carbonate, ferrous fumarate, folic acid, iodine, vitamin C, vitamin B6, vitamin B12, choline, vitamin E, vitamin D3, and D-biotin.

(34) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium carbonate, ferrous fumarate, folic acid, iodine, vitamin C, vitamin B6, vitamin B12, choline, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(35) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate calcium, calcium carbonate, ferrous fumarate, folic acid, iodine, vitamin C, vitamin B6, vitamin B12, choline, vitamin E, vitamin D3, D-biotin, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(36) A supplement comprising or consisting essentially of an omega-3 fatty acid, a folate derivative, calcium carbonate, ferrous fumarate, folic acid, vitamin C, vitamin B6, vitamin E, vitamin D3, D-biotin, and one or more of iodine, vitamin B12 and/or choline.

(37) A supplement comprising or consisting essentially of an omega-3 fatty acid, a folate derivative, docusate calcium, calcium carbonate, ferrous fumarate, folic acid, vitamin C, vitamin B6, vitamin E, vitamin D3, D-biotin, and one or more of iodine, vitamin B12 and/or choline.

(38) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), calcium carbonate, ferrous fumarate, folic acid, vitamin C, vitamin B6, vitamin E, vitamin D3, D-biotin, and one or more of iodine, vitamin B12 and/or choline, and further at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(39) A supplement comprising or consisting essentially of an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), docusate calcium, calcium carbonate, ferrous fumarate, folic acid, vitamin C, vitamin B6, vitamin E, vitamin D3, D-biotin, and one or more of iodine, vitamin B12 and/or choline, and further at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(40) A supplement comprising or consisting essentially of an omega-3 fatty acid, a folate derivative, and one or more of the following ingredients: calcium carbonate; ferrous fumarate; folic acid; vitamin A; vitamin C; vitamin D; vitamin E, vitamin B1; vitamin B2; vitamin B3; vitamin B5; vitamin B6; vitamin B 12; choline; vitamin D3; D-biotin; iodine; iron; zinc; copper; calcium; magnesium; an omega-3 fatty acid; and/or, any and all pharmaceutically acceptable forms of the foregoing.

(41) A supplement comprising or consisting essentially of an omega-3 fatty acid, a folate derivative, and one or more of the following ingredients: docusate calcium; calcium carbonate; ferrous fumarate; folic acid; vitamin A; vitamin C; vitamin D; vitamin E, vitamin B1; vitamin B2; vitamin B3; vitamin B5; vitamin B6; vitamin B12; choline; vitamin D3; D-biotin; iodine; iron; zinc; copper; calcium; magnesium; an omega-3 fatty acid; and/or, any and all pharmaceutically acceptable forms of the foregoing.

(42) A supplement comprising or consisting essentially of (i) an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), and one or more of the following ingredients: calcium carbonate; ferrous fumarate; folic acid; vitamin A; vitamin C; vitamin D; vitamin E, vitamin B1; vitamin B2; vitamin B3; vitamin B5; vitamin B6; vitamin B12; choline; vitamin D3; D-biotin; iodine; iron; zinc; copper; calcium; magnesium; an omega-3 fatty acid; and/or, any and all pharmaceutically acceptable forms of the foregoing; and, (2) at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(43) A supplement comprising or consisting essentially of (i) an omega-3 fatty acid (preferably, docosahexaenoic acid, and more preferably, docosahexaenoic acid of plant-based origin), a folate derivative (wherein the folate derivative is the glucosamine salt of (6S)-5-methyltetrahydrofolate), and one or more of the following ingredients: docusate calcium; calcium carbonate; ferrous fumarate; folic acid; vitamin A; vitamin C; vitamin D; vitamin E, vitamin B1; vitamin B2; vitamin B3; vitamin B5; vitamin B6; vitamin B12; choline; vitamin D3; D-biotin; iodine; iron; zinc; copper; calcium; magnesium; an omega-3 fatty acid; and/or, any and all pharmaceutically acceptable forms of the foregoing; and, (2) at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

### Examples of Specific Embodiments of the Present Invention

Examples of specific embodiments of the present invention include, but are not limited to, the following:

(1) A supplement comprising docosahexaenoic acid in an amount of about 350 mg to about 660.4 mg, calcium in an amount of about 157.5 mg to about 401.4 mg, iron in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(2) A supplement comprising docosahexaenoic acid in an amount of about 350 mg to about 660.4 mg, docusate in an amount of about 55 mg to about 110 mg, calcium in an amount of about 157.5 mg to about 401.4 mg, iron in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(3) A supplement comprising docosahexaenoic acid in an amount of about 350 mg to about 660.4 mg, calcium in an amount of about 157.5 mg to about 401.4 mg, iron in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(4) A supplement comprising docosahexaenoic acid in an amount of about 350 mg to about 660.4 mg, docusate in an amount of about 55 mg to about 110 mg, calcium in an amount of about 157.5 mg to about 401.4 mg, iron in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(5) A supplement comprising docosahexaenoic acid in an amount of about 350 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(6) A supplement comprising docosahexaenoic acid in an amount of about 350 mg, docusate in an amount of about 55 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(7) A supplement comprising docosahexaenoic acid in an amount of about 350 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(8) A supplement comprising docosahexaenoic acid in an amount of about 350 mg, docusate in an amount of about 55 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(9) A supplement comprising docosahexaenoic acid in an amount of about 660.4 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(10) A supplement comprising docosahexaenoic acid in an amount of about 660.4 mg, docusate in an amount of about 110 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(11) A supplement comprising docosahexaenoic acid in an amount of about 660.4 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(12) A supplement comprising docosahexaenoic acid in an amount of about 660.4 mg, docusate in an amount of about 110 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(13) A supplement consisting essentially of docosahexaenoic acid in an amount of about 350 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(14) A supplement consisting essentially of docosahexaenoic acid in an amount of about 350 mg, docusate in an amount of about 55 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(15) A supplement consisting essentially of docosahexaenoic acid in an amount of about 350 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(16) A supplement consisting essentially of docosahexaenoic acid in an amount of about 350 mg, docusate in an amount of about 55 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(17) A supplement consisting essentially of docosahexaenoic acid in an amount of about 660.4 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(18) A supplement consisting essentially of docosahexaenoic acid in an amount of about 660.4 mg, docusate in an amount of about 110 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(19) A supplement consisting essentially of docosahexaenoic acid in an amount of about 660.4 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(20) A supplement consisting essentially of docosahexaenoic acid in an amount of about 660.4 mg, docusate in an amount of about 110 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(21) A supplement comprising docosahexaenoic acid in an amount of about 350 mg to about 660.4 mg, calcium in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(22) A supplement comprising docosahexaenoic acid in an amount of about 350 mg to about 660.4 mg, docusate in an amount of about 55 mg to about 110 mg, calcium in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(23) A supplement comprising docosahexaenoic acid in an amount of about 350 mg to about 660.4 mg, calcium in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(24) A supplement comprising docosahexaenoic acid in an amount of about 350 mg to about 660.4 mg, docusate in an amount of about 55 mg to about 110 mg, calcium in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(25) A supplement comprising docosahexaenoic acid in an amount of about 350 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(26) A supplement comprising docosahexaenoic acid in an amount of about 350 mg, docusate in an amount of about 55 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(27) A supplement comprising docosahexaenoic acid in an amount of about 350 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(28) A supplement comprising docosahexaenoic acid in an amount of about 350 mg, docusate in an amount of about 55 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(29) A supplement comprising docosahexaenoic acid in an amount of about 660.4 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(30) A supplement comprising docosahexaenoic acid in an amount of about 660.4 mg, docusate in an amount of about 110 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(31) A supplement comprising docosahexaenoic acid in an amount of about 660.4 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(32) A supplement comprising docosahexaenoic acid in an amount of about 660.4 mg, docusate in an amount of about 110 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(33) A supplement consisting essentially of docosahexaenoic acid in an amount of about 350 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(34) A supplement consisting essentially of docosahexaenoic acid in an amount of about 350 mg, docusate in an amount of about 55 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(35) A supplement consisting essentially of docosahexaenoic acid in an amount of about 350 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(36) A supplement consisting essentially of docosahexaenoic acid in an amount of about 350 mg, docusate in an amount of about 55 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(37) A supplement consisting essentially of docosahexaenoic acid in an amount of about 660.4 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(38) A supplement consisting essentially of docosahexaenoic acid in an amount of about 660.4 mg, docusate in an amount of about 110 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(39) A supplement consisting essentially of docosahexaenoic acid in an amount of about 660.4 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(40) A supplement consisting essentially of docosahexaenoic acid in an amount of about 660.4 mg, docusate in an amount of about 110 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(41) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(42) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(43) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(44) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(45) A supplement comprising docosahexaenoic acid in an amount of about 350 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(46) A supplement comprising docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(47) A supplement comprising docosahexaenoic acid in an amount of about 350 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(48) A supplement comprising docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(49) A supplement comprising docosahexaenoic acid in an amount of about 660.4 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(50) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(51) A supplement comprising docosahexaenoic acid in an amount of about 660.4 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(52) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(53) A supplement consisting essentially of docosahexaenoic acid in an amount of about 350 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(54) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(55) A supplement consisting essentially of docosahexaenoic acid in an amount of about 350 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(56) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(57) A supplement consisting essentially of docosahexaenoic acid in an amount of about 660.4 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(58) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(59) A supplement consisting essentially of docosahexaenoic acid in an amount of about 660.4 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(60) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(61) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 0.6 mg to about 0.727 mg, (6S)-5-methyltetrahydrofolate glucosamine salt in an amount of about 1.2 mg to about 1.45 mg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(62) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 0.6 mg to about 0.727 mg, (6S)-5-methyltetrahydrofolate glucosamine salt in an amount of about 1.2 mg to about 1.45 mg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(63) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 0.6 mg to about 0.727 mg, (6S)-5-methyltetrahydrofolate glucosamine salt in an amount of about 1.2 mg to about 1.45 mg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(64) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 0.6 mg to about 0.727 mg, (6S)-5-methyltetrahydrofolate glucosamine salt in an amount of about 1.2 mg to about 1.45 mg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(65) A supplement comprising docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(66) A supplement comprising docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(67) A supplement comprising docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(68) A supplement comprising docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(69) A supplement comprising docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(70) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(71) A supplement comprising docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(72) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(73) A supplement consisting essentially of docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(74) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(75) A supplement consisting essentially of docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(76) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(77) A supplement consisting essentially of docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(78) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(79) A supplement consisting essentially of docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(80) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(81) A supplement comprising docosahexaenoic acid in an amount of about 350 mg to about 660.4 mg, calcium in an amount of about 157.5 mg to about 401.4 mg, iron in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(82) A supplement comprising docosahexaenoic acid in an amount of about 350 mg to about 660.4 mg, docusate in an amount of about 55 mg to about 110 mg, calcium in an amount of about 157.5 mg to about 401.4 mg, iron in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(83) A supplement comprising docosahexaenoic acid in an amount of about 350 mg to about 660.4 mg, calcium in an amount of about 157.5 mg to about 401.4 mg, iron in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(84) A supplement comprising docosahexaenoic acid in an amount of about 350 mg to about 660.4 mg, docusate in an amount of about 55 mg to about 110 mg, calcium in an amount of about 157.5 mg to about 401.4 mg, iron in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(85) A supplement comprising docosahexaenoic acid in an amount of about 350 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(86) A supplement comprising docosahexaenoic acid in an amount of about 350 mg, docusate in an amount of about 55 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(87) A supplement comprising docosahexaenoic acid in an amount of about 350 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(88) A supplement comprising docosahexaenoic acid in an amount of about 350 mg, docusate in an amount of about 55 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(89) A supplement comprising docosahexaenoic acid in an amount of about 660.4 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(90) A supplement comprising docosahexaenoic acid in an amount of about 660.4 mg, docusate in an amount of about 110 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(91) A supplement comprising docosahexaenoic acid in an amount of about 660.4 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(92) A supplement comprising docosahexaenoic acid in an amount of about 660.4 mg, docusate in an amount of about 110 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(93) A supplement consisting essentially of docosahexaenoic acid in an amount of about 350 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(94) A supplement consisting essentially of docosahexaenoic acid in an amount of about 350 mg, docusate in an amount of about 55 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(95) A supplement consisting essentially of docosahexaenoic acid in an amount of about 350 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(96) A supplement consisting essentially of docosahexaenoic acid in an amount of about 350 mg, docusate in an amount of about 55 mg, calcium in an amount of about 157.5 mg, iron in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(97) A supplement consisting essentially of docosahexaenoic acid in an amount of about 660.4 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(98) A supplement consisting essentially of docosahexaenoic acid in an amount of about 660.4 mg, docusate in an amount of about 110 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(99) A supplement consisting essentially of docosahexaenoic acid in an amount of about 660.4 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(100) A supplement consisting essentially of docosahexaenoic acid in an amount of about 660.4 mg, docusate in an amount of about 110 mg, calcium in an amount of about 401.4 mg, iron in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(101) A supplement comprising docosahexaenoic acid in an amount of about 350 mg to about 660.4 mg, calcium in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(102) A supplement comprising docosahexaenoic acid in an amount of about 350 mg to about 660.4 mg, docusate in an amount of about 55 mg to about 110 mg, calcium in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(103) A supplement comprising docosahexaenoic acid in an amount of about 350 mg to about 660.4 mg, calcium in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(104) A supplement comprising docosahexaenoic acid in an amount of about 350 mg to about 660.4 mg, docusate in an amount of about 55 mg to about 110 mg, calcium in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(105) A supplement comprising docosahexaenoic acid in an amount of about 350 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(106) A supplement comprising docosahexaenoic acid in an amount of about 350 mg, docusate in an amount of about 55 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(107) A supplement comprising docosahexaenoic acid in an amount of about 350 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(108) A supplement comprising docosahexaenoic acid in an amount of about 350 mg, docusate in an amount of about 55 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(109) A supplement comprising docosahexaenoic acid in an amount of about 660.4 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(110) A supplement comprising docosahexaenoic acid in an amount of about 660.4 mg, docusate in an amount of about 110 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(111) A supplement comprising docosahexaenoic acid in an amount of about 660.4 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(112) A supplement comprising docosahexaenoic acid in an amount of about 660.4 mg, docusate in an amount of about 110 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(113) A supplement consisting essentially of docosahexaenoic acid in an amount of about 350 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(114) A supplement consisting essentially of docosahexaenoic acid in an amount of about 350 mg, docusate in an amount of about 55 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(115) A supplement consisting essentially of docosahexaenoic acid in an amount of about 350 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(116) A supplement consisting essentially of docosahexaenoic acid in an amount of about 350 mg, docusate in an amount of about 55 mg, calcium in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(117) A supplement consisting essentially of docosahexaenoic acid in an amount of about 660.4 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(118) A supplement consisting essentially of docosahexaenoic acid in an amount of about 660.4 mg, docusate in an amount of about 110 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(119) A supplement consisting essentially of docosahexaenoic acid in an amount of about 660.4 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(120) A supplement consisting essentially of docosahexaenoic acid in an amount of about 660.4 mg, docusate in an amount of about 110 mg, calcium in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(121) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(122) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(123) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(124) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, folic acid in an amount of about 0.6 mg to about 0.727 mg, (6S)-5-methyltetrahydrofolate glucosamine salt in an amount of about 1.2 mg to about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(125) A supplement comprising docosahexaenoic acid in an amount of about 350 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(126) A supplement comprising docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(127) A supplement comprising docosahexaenoic acid in an amount of about 350 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(128) A supplement comprising docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(129) A supplement comprising docosahexaenoic acid in an amount of about 660.4 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(130) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(131) A supplement comprising docosahexaenoic acid in an amount of about 660.4 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(132) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(133) A supplement consisting essentially of docosahexaenoic acid in an amount of about 350 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(134) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(135) A supplement consisting essentially of docosahexaenoic acid in an amount of about 350 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(136) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 0.6 mg, (6S)-5-methyltetrahydrofolate glucosamine salt in an amount of about 1.2 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(137) A supplement consisting essentially of docosahexaenoic acid in an amount of about 660.4 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(138) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(139) A supplement consisting essentially of docosahexaenoic acid in an amount of about 660.4 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(140) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 0.727 mg, (6S)-5-methyltetrahydrofolate glucosamine salt in an amount of about 1.45 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(141) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 0.6 mg to about 0.727 mg, (6S)-5-methyltetrahydrofolate glucosamine salt in an amount of about 1.2 mg to about 1.45 mg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(142) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 0.6 mg to about 0.727 mg, (6S)-5-methyltetrahydrofolate glucosamine salt in an amount of about 1.2 mg to about 1.45 mg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(143) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 0.6 mg to about 0.727 mg, (6S)-5-methyltetrahydrofolate glucosamine salt in an amount of about 1.2 mg to about 1.45 mg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(144) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 0.6 mg to about 0.727 mg, (6S)-5-methyltetrahydrofolate glucosamine salt in an amount of about 1.2 mg to about 1.45 mg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(145) A supplement comprising docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(146) A supplement comprising docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(147) A supplement comprising docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(148) A supplement comprising docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(149) A supplement comprising docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(150) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(151) A supplement comprising docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(152) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(153) A supplement consisting essentially of docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(154) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(155) A supplement consisting essentially of docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(156) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 0.6 mg, (6S)-5-methyltetrahydrofolate glucosamine salt in an amount of about 1.2 mg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(157) A supplement consisting essentially of docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(158) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(159) A supplement consisting essentially of docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(160) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 0.727 mg, (6S)-5-methyltetrahydrofolate glucosamine salt in an amount of about 1.45 mg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(161) A supplement comprising docosahexaenoic acid in an amount of about 350 mg to about 660.4 mg, calcium in an amount of about 157.5 mg to about 401.4 mg, iron in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(162) A supplement comprising docosahexaenoic acid in an amount of about 350 mg to about 660.4 mg, docusate in an amount of about 55 mg to about 110 mg, calcium in an amount of about 157.5 mg to about 401.4 mg, iron in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. (163) A supplement comprising docosahexaenoic acid in an amount of about 350 mg to about 660.4 mg, calcium in an amount of about 157.5 mg to about 401.4 mg, iron in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(164) A supplement comprising docosahexaenoic acid in an amount of about 350 mg to about 660.4 mg, docusate in an amount of about 55 mg to about 110 mg, calcium in an amount of about 157.5 mg to about 401.4 mg, iron in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(165) A supplement comprising docosahexaenoic acid in an amount of about 350 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. (166) A supplement comprising docosahexaenoic acid in an amount of about 350 mg, docusate in an amount of about 55 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(167) A supplement comprising docosahexaenoic acid in an amount of about 350 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(168) A supplement comprising docosahexaenoic acid in an amount of about 350 mg, docusate in an amount of about 55 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(169) A supplement comprising docosahexaenoic acid in an amount of about 660.4 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(170) A supplement comprising docosahexaenoic acid in an amount of about 660.4 mg, docusate in an amount of about 110 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(171) A supplement comprising docosahexaenoic acid in an amount of about 660.4 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(172) A supplement comprising docosahexaenoic acid in an amount of about 660.4 mg, docusate in an amount of about 110 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(173) A supplement consisting essentially of docosahexaenoic acid in an amount of about 350 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(174) A supplement consisting essentially of docosahexaenoic acid in an amount of about 350 mg, docusate in an amount of about 55 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(175) A supplement consisting essentially of docosahexaenoic acid in an amount of about 350 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(176) A supplement consisting essentially of docosahexaenoic acid in an amount of about 350 mg, docusate in an amount of about 55 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(177) A supplement consisting essentially of docosahexaenoic acid in an amount of about 660.4 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(178) A supplement consisting essentially of docosahexaenoic acid in an amount of about 660.4 mg, docusate in an amount of about 110 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(179) A supplement consisting essentially of docosahexaenoic acid in an amount of about 660.4 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(180) A supplement consisting essentially of docosahexaenoic acid in an amount of about 660.4 mg, docusate in an amount of about 110 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(181) A supplement comprising docosahexaenoic acid in an amount of about 350 mg to about 660.4 mg, calcium in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(182) A supplement comprising docosahexaenoic acid in an amount of about 350 mg to about 660.4 mg, docusate in an amount of about 55 mg to about 110 mg, calcium in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(183) A supplement comprising docosahexaenoic acid in an amount of about 350 mg to about 660.4 mg, calcium in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(184) A supplement comprising docosahexaenoic acid in an amount of about 350 mg to about 660.4 mg, docusate in an amount of about 55 mg to about 110 mg, calcium in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(185) A supplement comprising docosahexaenoic acid in an amount of about 350 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(186) A supplement comprising docosahexaenoic acid in an amount of about 350 mg, docusate in an amount of about 55 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(187) A supplement comprising docosahexaenoic acid in an amount of about 350 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(188) A supplement comprising docosahexaenoic acid in an amount of about 350 mg, docusate in an amount of about 55 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(189) A supplement comprising docosahexaenoic acid in an amount of about 660.4 mg, calcium in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(190) A supplement comprising docosahexaenoic acid in an amount of about 660.4 mg, docusate in an amount of about 110 mg, calcium in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(191) A supplement comprising docosahexaenoic acid in an amount of about 660.4 mg, calcium in an amount of about 401.4 mg, ferrous fumarate in an amount of about 91 mg, folic acid in an amount of to about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(192) A supplement comprising docosahexaenoic acid in an amount of about 660.4 mg, docusate in an amount of about 110 mg, calcium in an amount of about 401.4 mg, ferrous fumarate in an amount of about 91 mg, folic acid in an amount of to about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(193) A supplement consisting essentially of docosahexaenoic acid in an amount of about 350 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(194) A supplement consisting essentially of docosahexaenoic acid in an amount of about 350 mg, docusate in an amount of about 55 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(195) A supplement consisting essentially of docosahexaenoic acid in an amount of about 350 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(196) A supplement consisting essentially of docosahexaenoic acid in an amount of about 350 mg, docusate in an amount of about 55 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(197) A supplement consisting essentially of docosahexaenoic acid in an amount of about 660.4 mg, calcium in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(198) A supplement consisting essentially of docosahexaenoic acid in an amount of about 660.4 mg, docusate in an amount of about 110 mg, calcium in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(199) A supplement consisting essentially of docosahexaenoic acid in an amount of about 660.4 mg, calcium in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(200) A supplement consisting essentially of docosahexaenoic acid in an amount of about 660.4 mg, docusate in an amount of about 110 mg, calcium in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(201) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(202) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(203) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(204) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(205) A supplement comprising docosahexaenoic acid in an amount of about 350 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(206) A supplement comprising docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(207) A supplement comprising docosahexaenoic acid in an amount of about 350 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(208) A supplement comprising docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(209) A supplement comprising docosahexaenoic acid in an amount of about 660.4 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(210) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(211) A supplement comprising docosahexaenoic acid in an amount of about 660.4 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(212) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(213) A supplement consisting essentially of docosahexaenoic acid in an amount of about 350 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(214) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(215) A supplement consisting essentially of docosahexaenoic acid in an amount of about 350 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(216) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(217) A supplement consisting essentially of docosahexaenoic acid in an amount of about 660.4 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(218) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(219) A supplement consisting essentially of docosahexaenoic acid in an amount of about 660.4 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(220) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(221) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 800 mcg to about 1 mg, (6S)-5-methyltetrahydrofolate glucosamine salt in an amount of about 550 mcg to about 750 mcg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(222) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 800 mcg to about 1 mg, (6S)-5-methyltetrahydrofolate glucosamine salt in an amount of about 550 mcg to about 750 mcg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(223) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 800 mcg to about 1 mg, (6S)-5-methyltetrahydrofolate glucosamine salt in an amount of about 550 mcg to about 750 mcg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(224) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 800 mcg to about 1 mg, (6S)-5-methyltetrahydrofolate glucosamine salt in an amount of about 550 mcg to about 750 mcg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(225) A supplement comprising docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(226) A supplement comprising docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(227) A supplement comprising docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(228) A supplement comprising docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(229) A supplement comprising docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(230) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(231) A supplement comprising docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(232) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(233) A supplement consisting essentially of docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(234) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(235) A supplement consisting essentially of docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(236) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(237) A supplement consisting essentially of docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(238) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(239) A supplement consisting essentially of docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(240) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(241) A supplement comprising docosahexaenoic acid in an amount of about 350 mg to about 660.4 mg, calcium in an amount of about 157.5 mg to about 401.4 mg, iron in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(242) A supplement comprising docosahexaenoic acid in an amount of about 350 mg to about 660.4 mg, docusate in an amount of about 55 mg to about 110 mg, calcium in an amount of about 157.5 mg to about 401.4 mg, iron in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(243) A supplement comprising docosahexaenoic acid in an amount of about 350 mg to about 660.4 mg, calcium in an amount of about 157.5 mg to about 401.4 mg, iron in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(244) A supplement comprising docosahexaenoic acid in an amount of about 350 mg to about 660.4 mg, docusate in an amount of about 55 mg to about 110 mg, calcium in an amount of about 157.5 mg to about 401.4 mg, iron in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(245) A supplement comprising docosahexaenoic acid in an amount of about 350 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(246) A supplement comprising docosahexaenoic acid in an amount of about 350 mg, docusate in an amount of about 55 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(247) A supplement comprising docosahexaenoic acid in an amount of about 350 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(248) A supplement comprising docosahexaenoic acid in an amount of about 350 mg, docusate in an amount of about 55 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(249) A supplement comprising docosahexaenoic acid in an amount of about 660.4 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(250) A supplement comprising docosahexaenoic acid in an amount of about 660.4 mg, docusate in an amount of about 110 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(251) A supplement comprising docosahexaenoic acid in an amount of about 660.4 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(252) A supplement comprising docosahexaenoic acid in an amount of about 660.4 mg, docusate in an amount of about 110 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(253) A supplement consisting essentially of docosahexaenoic acid in an amount of about 350 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(254) A supplement consisting essentially of docosahexaenoic acid in an amount of about 350 mg, docusate in an amount of about 55 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(255) A supplement consisting essentially of docosahexaenoic acid in an amount of about 350 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(256) A supplement consisting essentially of docosahexaenoic acid in an amount of about 350 mg, docusate in an amount of about 55 mg, calcium in an amount of about 157.5 mg, iron in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(257) A supplement consisting essentially of docosahexaenoic acid in an amount of about 660.4 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(258) A supplement consisting essentially of docosahexaenoic acid in an amount of about 660.4 mg, docusate in an amount of about 110 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(259) A supplement consisting essentially of docosahexaenoic acid in an amount of about 660.4 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(260) A supplement consisting essentially of docosahexaenoic acid in an amount of about 660.4 mg, docusate in an amount of about 110 mg, calcium in an amount of about 401.4 mg, iron in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(261) A supplement comprising docosahexaenoic acid in an amount of about 350 mg to about 660.4 mg, calcium in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(262) A supplement comprising docosahexaenoic acid in an amount of about 350 mg to about 660.4 mg, docusate in an amount of about 55 mg to about 110 mg, calcium in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(263) A supplement comprising docosahexaenoic acid in an amount of about 350 mg to about 660.4 mg, calcium in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(264) A supplement comprising docosahexaenoic acid in an amount of about 350 mg to about 660.4 mg, docusate in an amount of about 55 mg to about 110 mg, calcium in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(265) A supplement comprising docosahexaenoic acid in an amount of about 350 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(266) A supplement comprising docosahexaenoic acid in an amount of about 350 mg, docusate in an amount of about 55 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(267) A supplement comprising docosahexaenoic acid in an amount of about 350 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(268) A supplement comprising docosahexaenoic acid in an amount of about 350 mg, docusate in an amount of about 55 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(269) A supplement comprising docosahexaenoic acid in an amount of about 660.4 mg, calcium in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(270) A supplement comprising docosahexaenoic acid in an amount of about 660.4 mg, docusate in an amount of about 110 mg, calcium in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(271) A supplement comprising docosahexaenoic acid in an amount of about 660.4 mg, calcium in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(272) A supplement comprising docosahexaenoic acid in an amount of about 660.4 mg, docusate in an amount of about 110 mg, calcium in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(273) A supplement consisting essentially of docosahexaenoic acid in an amount of about 350 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(274) A supplement consisting essentially of docosahexaenoic acid in an amount of about 350 mg, docusate in an amount of about 55 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(275) A supplement consisting essentially of docosahexaenoic acid in an amount of about 350 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(276) A supplement consisting essentially of docosahexaenoic acid in an amount of about 350 mg, docusate in an amount of about 55 mg, calcium in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(277) A supplement consisting essentially of docosahexaenoic acid in an amount of about 660.4 mg, calcium in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(278) A supplement consisting essentially of docosahexaenoic acid in an amount of about 660.4 mg, docusate in an amount of about 110 mg, calcium in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(279) A supplement consisting essentially of docosahexaenoic acid in an amount of about 660.4 mg, calcium in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(280) A supplement consisting essentially of docosahexaenoic acid in an amount of about 660.4 mg, docusate in an amount of about 110 mg, calcium in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(281) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(282) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(283) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(284) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, folic acid in an amount of about 800 mcg to about 1 mg, (6S)-5-methyltetrahydrofolate glucosamine salt in an amount of about 550 mcg to about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(285) A supplement comprising docosahexaenoic acid in an amount of about 350 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(286) A supplement comprising docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(287) A supplement comprising docosahexaenoic acid in an amount of about 350 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(288) A supplement comprising docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(289) A supplement comprising docosahexaenoic acid in an amount of about 660.4 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(290) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(291) A supplement comprising docosahexaenoic acid in an amount of about 660.4 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(292) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(293) A supplement consisting essentially of docosahexaenoic acid in an amount of about 350 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(294) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(295) A supplement consisting essentially of docosahexaenoic acid in an amount of about 350 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(296) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 800 mcg, (6S)-5-methyltetrahydrofolate glucosamine salt in an amount of about 550 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(297) A supplement consisting essentially of docosahexaenoic acid in an amount of about 660.4 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(298) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(299) A supplement consisting essentially of docosahexaenoic acid in an amount of about 660.4 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(300) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, folic acid in an amount of about 1 mg, (6S)-5-methyltetrahydrofolate glucosamine salt in an amount of about 750 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(301) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 800 mcg to about 1 mg, (6S)-5-methyltetrahydrofolate glucosamine salt in an amount of about 550 mcg to about 750 mcg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(302) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 800 mcg to about 1 mg, (6S)-5-methyltetrahydrofolate glucosamine salt in an amount of about 550 mcg to about 750 mcg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(303) A supplement comprising docosahexaenoic acid in an amount of about 350 mg to about 660.4 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 800 mcg to about 1 mg, (6S)-5-methyltetrahydrofolate glucosamine salt in an amount of about 550 mcg to about 750 mcg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(304) A supplement comprising docosahexaenoic acid in an amount of about 350 mg to about 660.4 mg, docusate calcium in an amount of about 55 mg to about 110 mg, calcium carbonate in an amount of about 157.5 mg to about 401.4 mg, ferrous fumarate in an amount of about 29 mg to about 91 mg, vitamin C in an amount of about 28 mg to about 42.9 mg, vitamin B6 in an amount of about 26 mg to about 37.9 mg, vitamin E in an amount of about 20.13 mg to about 30 mg, folic acid in an amount of about 800 mcg to about 1 mg, (6S)-5-methyltetrahydrofolate glucosamine salt in an amount of about 550 mcg to about 750 mcg, vitamin D3 in an amount of about 20.625 mcg to about 0.99 mg, D-biotin in an amount of about 250 mcg to about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(305) A supplement comprising docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(306) A supplement comprising docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(307) A supplement comprising docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(308) A supplement comprising docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(309) A supplement comprising docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(310) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(311) A supplement comprising docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(312) A supplement comprising docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(313) A supplement consisting essentially of docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(314) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(315) A supplement consisting essentially of docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(316) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 800 mcg, (6S)-5-methyltetrahydrofolate glucosamine salt in an amount of about 550 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(317) A supplement consisting essentially of docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(318) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(319) A supplement consisting essentially of docosahexaenoic acid in an amount of about 660.4 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(320) A supplement consisting essentially of docosahexaenoic acid in an amount of about 660.4 mg, docusate calcium in an amount of about 110 mg, calcium carbonate in an amount of about 401.4 mg, ferrous fumarate in an amount of about 91 mg, vitamin C in an amount of about 42.9 mg, vitamin B6 in an amount of about 37.9 mg, vitamin E in an amount of about 30 mg, folic acid in an amount of about 1 mg, (6S)-5-methyltetrahydrofolate glucosamine salt in an amount of about 750 mcg, vitamin D3 in an amount of about 0.99 mg, D-biotin in an amount of about 0.317 mg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(321) A supplement comprising docosahexaenoic acid in an amount of about 350 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(322) A supplement comprising docosahexaenoic acid in an amount of about 350 mg, docusate in an amount of about 55 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(323) A supplement comprising docosahexaenoic acid in an amount of about 350 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(324) A supplement comprising docosahexaenoic acid in an amount of about 350 mg, docusate in an amount of about 55 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(325) A supplement consisting essentially of docosahexaenoic acid in an amount of about 350 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(326) A supplement consisting essentially of docosahexaenoic acid in an amount of about 350 mg, docusate in an amount of about 55 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(327) A supplement consisting essentially of docosahexaenoic acid in an amount of about 350 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(328) A supplement consisting essentially of docosahexaenoic acid in an amount of about 350 mg, docusate in an amount of about 55 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(329) A supplement comprising docosahexaenoic acid in an amount of about 350 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(330) A supplement comprising docosahexaenoic acid in an amount of about 350 mg, docusate in an amount of about 55 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(331) A supplement comprising docosahexaenoic acid in an amount of about 350 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(332) A supplement comprising docosahexaenoic acid in an amount of about 350 mg, docusate in an amount of about 55 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(333) A supplement consisting essentially of docosahexaenoic acid in an amount of about 350 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(334) A supplement consisting essentially of docosahexaenoic acid in an amount of about 350 mg, docusate in an amount of about 55 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(335) A supplement consisting essentially of docosahexaenoic acid in an amount of about 350 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(336) A supplement consisting essentially of docosahexaenoic acid in an amount of about 350 mg, docusate in an amount of about 55 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(337) A supplement comprising docosahexaenoic acid in an amount of about 350 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(338) A supplement comprising docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(339) A supplement comprising docosahexaenoic acid in an amount of about 350 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(340) A supplement comprising docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(341) A supplement consisting essentially of docosahexaenoic acid in an amount of about 350 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(342) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(343) A supplement consisting essentially of docosahexaenoic acid in an amount of about 350 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(344) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(345) A supplement comprising docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(346) A supplement comprising docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(347) A supplement comprising docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(348) A supplement comprising docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(349) A supplement consisting essentially of docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(350) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

(351) A supplement consisting essentially of docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(352) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(353) A supplement comprising docosahexaenoic acid in an amount of about 350 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(354) A supplement comprising docosahexaenoic acid in an amount of about 350 mg, docusate in an amount of about 55 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(355) A supplement comprising docosahexaenoic acid in an amount of about 350 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(356) A supplement comprising docosahexaenoic acid in an amount of about 350 mg, docusate in an amount of about 55 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(357) A supplement consisting essentially of docosahexaenoic acid in an amount of about 350 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(358) A supplement consisting essentially of docosahexaenoic acid in an amount of about 350 mg, docusate in an amount of about 55 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(359) A supplement consisting essentially of docosahexaenoic acid in an amount of about 350 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(360) A supplement consisting essentially of docosahexaenoic acid in an amount of about 350 mg, docusate in an amount of about 55 mg, calcium in an amount of about 157.5 mg, iron in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(361) A supplement comprising docosahexaenoic acid in an amount of about 350 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(362) A supplement comprising docosahexaenoic acid in an amount of about 350 mg, docusate in an amount of about 55 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(363) A supplement comprising docosahexaenoic acid in an amount of about 350 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(364) A supplement comprising docosahexaenoic acid in an amount of about 350 mg, docusate in an amount of about 55 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(365) A supplement consisting essentially of docosahexaenoic acid in an amount of about 350 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(366) A supplement consisting essentially of docosahexaenoic acid in an amount of about 350 mg, docusate in an amount of about 55 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(367) A supplement consisting essentially of docosahexaenoic acid in an amount of about 350 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(368) A supplement consisting essentially of docosahexaenoic acid in an amount of about 350 mg, docusate in an amount of about 55 mg, calcium in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(369) A supplement comprising docosahexaenoic acid in an amount of about 350 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(370) A supplement comprising docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(371) A supplement comprising docosahexaenoic acid in an amount of about 350 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(372) A supplement comprising docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(373) A supplement consisting essentially of docosahexaenoic acid in an amount of about 350 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(374) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(375) A supplement consisting essentially of docosahexaenoic acid in an amount of about 350 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(376) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, folic acid in an amount of about 950 mcg, (6S)-5-methyltetrahydrofolate glucosamine salt in an amount of about 555.5 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(377) A supplement comprising docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(378) A supplement comprising docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(379) A supplement comprising docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(380) A supplement comprising docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(381) A supplement consisting essentially of docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(382) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(383) A supplement consisting essentially of docosahexaenoic acid in an amount of about 350 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(384) A supplement consisting essentially of docosahexaenoic acid in an amount of about 350 mg, docusate calcium in an amount of about 55 mg, calcium carbonate in an amount of about 157.5 mg, ferrous fumarate in an amount of about 29 mg, vitamin C in an amount of about 28 mg, vitamin B6 in an amount of about 26 mg, vitamin E in an amount of about 20.13 mg, folic acid in an amount of about 950 mcg, (6S)-5-methyltetrahydrofolate glucosamine salt in an amount of about 555.5 mcg, vitamin D3 in an amount of about 20.625 mcg, D-biotin in an amount of about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(385) A supplement comprising docosahexaenoic acid in an amount of at least about 350 mg, docusate calcium in an amount of at least about 55 mg, calcium in an amount of at least about 160 mg (wherein at least about 157.5 mg of calcium is in the form of calcium carbonate and, optionally, wherein at least about 2 mg of calcium is provided by the at least about 55 mg of docusate calcium), ferrous fumarate in an amount of at least about 29 mg, vitamin C in an amount of at least about 28 mg, vitamin B6 in an amount of at least about 26 mg, vitamin E in an amount of at least about 20.13 mg, folic acid in an amount of at least about 0.6 mg, (6S)-5-methyltetrahydrofolate glucosamine salt in an amount of at least about 1.2 mg, vitamin D3 in an amount of at least about 20.625 mcg, D-biotin in an amount of at least about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule.

(386) A supplement comprising docosahexaenoic acid in an amount of at least about 350 mg, docusate calcium in an amount of at least about 55 mg, calcium in an amount of at least about 160 mg (wherein at least about 157.5 mg of calcium is in the form of calcium carbonate and, optionally, wherein at least about 2 mg of calcium is provided by the at least about 55 mg of docusate calcium), ferrous fumarate in an amount of at least about 29 mg, vitamin C in an amount of at least about 28 mg, vitamin B6 in an amount of at least about 26 mg, vitamin E in an amount of at least about 20.13 mg, folic acid in an amount of at least about 0.6 mg, (6S)-5-methyltetrahydrofolate glucosamine salt in an amount of at least about 1.2 mg, vitamin D3 in an amount of at least about 20.625 mcg, D-biotin in an amount of at least about 250 mcg, and at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks, and wherein the supplement is provided in the dosage form of a swallowable, single softgel capsule. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, (6S)-5-methyltetrahydrofolate glucosamine salt is preferably of the form available under the Quatrefolic® trademark, and assigned CAS Number 1181972-37-1.

(387) A supplement for administration to a human, wherein the supplement comprises a softgel capsule comprising ingredients in amounts sufficient to provide a human with at least about 350 mg of docosahexaenoic acid, at least about 157.5 mg of calcium carbonate, at least about 29 mg of ferrous fumarate, at least about 28 mg of vitamin C, at least about 26 mg of vitamin B6, at least about 20.13 mg of vitamin E, at least about 0.6 mg of folic acid, at least about 1.2 mg of a folate derivative, at least about 20.625 mcg of vitamin D3, and at least about 250 mcg of D-biotin.

(388) A supplement for administration to a human, wherein the supplement comprises a softgel capsule comprising ingredients in amounts sufficient to provide a human with at least about 350 mg of docosahexaenoic acid, at least about 55 mg of docusate calcium, at least about 157.5 mg of calcium carbonate, at least about 29 mg of ferrous fumarate, at least about 28 mg of vitamin C, at least about 26 mg of vitamin B6, at least about 20.13 mg of vitamin E, at least about 0.6 mg of folic acid, at least about 1.2 mg of a folate derivative, at least about 20.625 mcg of vitamin D3, and at least about 250 mcg of D-biotin.

(389) A supplement for administration to a human, wherein the supplement comprises a softgel capsule comprising ingredients in amounts sufficient to provide a human with at least about 660.4 mg of docosahexaenoic acid, at least about 401.4 mg of calcium carbonate, at least about 91 mg of ferrous fumarate, at least about 42.9 mg of vitamin C, at least about 37.9 mg of vitamin B6, at least about 30 mg of vitamin E, at least about 0.727 mg of folic acid, at least about 1.45 mg of a folate derivative, at least about 0.99 mg of vitamin D3, and at least about 0.317 mg of D-biotin.

(390) A supplement for administration to a human, wherein the supplement comprises a softgel capsule comprising ingredients in amounts sufficient to provide a human with at least about 660.4 mg of docosahexaenoic acid, at least about 110 mg of docusate calcium, at least about 401.4 mg of calcium carbonate, at least about 91 mg of ferrous fumarate, at least about 42.9 mg of vitamin C, at least about 37.9 mg of vitamin B6, at least about 30 mg of vitamin E, at least about 0.727 mg of folic acid, at least about 1.45 mg of a folate derivative, at least about 0.99 mg of vitamin D3, and at least about 0.317 mg of D-biotin.

(391) A supplement for administration to a human, wherein the supplement comprises a softgel capsule comprising ingredients in amounts sufficient to provide a human with at least about 350 mg of docosahexaenoic acid, at least about 157.5 mg of calcium carbonate, at least about 29 mg of ferrous fumarate, at least about 28 mg of vitamin C, at least about 26 mg of vitamin B6, at least about 20.13 mg of vitamin E, at least about 800 mcg of folic acid, at least about 550 mcg of a folate derivative, at least about 20.625 mcg of vitamin D3, and at least about 250 mcg of D-biotin.

(392) A supplement for administration to a human, wherein the supplement comprises a softgel capsule comprising ingredients in amounts sufficient to provide a human with at least about 350 mg of docosahexaenoic acid, at least about 55 mg of docusate calcium, at least about 157.5 mg of calcium carbonate, at least about 29 mg of ferrous fumarate, at least about 28 mg of vitamin C, at least about 26 mg of vitamin B6, at least about 20.13 mg of vitamin E, at least about 800 mcg of folic acid, at least about 550 mcg of a folate derivative, at least about 20.625 mcg of vitamin D3, and at least about 250 mcg of D-biotin.

(393) A supplement for administration to a human, wherein the supplement comprises a softgel capsule comprising ingredients in amounts sufficient to provide a human with at least about 660.4 mg of docosahexaenoic acid, at least about 401.4 mg of calcium carbonate, at least about 91 mg of ferrous fumarate, at least about 42.9 mg of vitamin C, at least about 37.9 mg of vitamin B6, at least about 30 mg of vitamin E, at least about 1 mg of folic acid, at least about 750 mcg of a folate derivative, at least about 0.99 mg of vitamin D3, and at least about 0.317 mg of D-biotin.

(394) A supplement for administration to a human, wherein the supplement comprises a softgel capsule comprising ingredients in amounts sufficient to provide a human with at least about 660.4 mg of docosahexaenoic acid, at least about 110 mg of docusate calcium, at least about 401.4 mg of calcium carbonate, at least about 91 mg of ferrous fumarate, at least about 42.9 mg of vitamin C, at least about 37.9 mg of vitamin B6, at least about 30 mg of vitamin E, at least about 1 mg of folic acid, at least about 750 mcg of a folate derivative, at least about 0.99 mg of vitamin D3, and at least about 0.317 mg of D-biotin.

(395) A supplement for administration to a human, wherein the supplement comprises a softgel capsule comprising ingredients in amounts sufficient to provide a human with at least about 350 mg of docosahexaenoic acid, at least about 157.5 mg of calcium carbonate, at least about 29 mg of ferrous fumarate, at least about 28 mg of vitamin C, at least about 26 mg of vitamin B6, at least about 20.13 mg of vitamin E, at least about 950 mcg of folic acid, at least about 555.5 mcg of a folate derivative, at least about 20.625 mcg of vitamin D3, and at least about 250 mcg of D-biotin.

(396) A supplement for administration to a human, wherein the supplement comprises a softgel capsule comprising ingredients in amounts sufficient to provide a human with at least about 350 mg of docosahexaenoic acid, at least about 55 mg of docusate calcium, at least about 157.5 mg of calcium carbonate, at least about 29 mg of ferrous fumarate, at least about 28 mg of vitamin C, at least about 26 mg of vitamin B6, at least about 20.13 mg of vitamin E, at least about 950 mcg of folic acid, at least about 555.5 mcg of a folate derivative, at least about 20.625 mcg of vitamin D3, and at least about 250 mcg of D-biotin.

(397) A supplement for administration to a human, wherein the supplement comprises a softgel capsule comprising ingredients in amounts sufficient to provide a human with at least about 350 mg of docosahexaenoic acid, at least about 157.5 mg of calcium carbonate, at least about 29 mg of ferrous fumarate, at least about 28 mg of vitamin C, at least about 26 mg of vitamin B6, at least about 20.13 mg of vitamin E, at least about 0.6 mg of folic acid, at least about 1.2 mg of a folate derivative, at least about 20.625 mcg of vitamin D3, and at least about 250 mcg of D-biotin. The foregoing specific embodiment may further comprise at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, the folate derivative is preferable in the form of (6S)-5-methyltetrahydrofolate glucosamine salt of the form available under the Quatrefolic® trademark, and assigned CAS Number 1181972-37-1.

(398) A supplement for administration to a human, wherein the supplement comprises a softgel capsule comprising ingredients in amounts sufficient to provide a human with at least about 350 mg of docosahexaenoic acid, at least about 55 mg of docusate calcium, at least about 157.5 mg of calcium carbonate, at least about 29 mg of ferrous fumarate, at least about 28 mg of vitamin C, at least about 26 mg of vitamin B6, at least about 20.13 mg of vitamin E, at least about 0.6 mg of folic acid, at least about 1.2 mg of a folate derivative, at least about 20.625 mcg of vitamin D3, and at least about 250 mcg of D-biotin. The foregoing specific embodiment may further comprise at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, the folate derivative is preferable in the form of (6S)-5-methyltetrahydrofolate glucosamine salt of the form available under the Quatrefolic® trademark, and assigned CAS Number 1181972-37-1.

(399) A supplement for administration to a human, wherein the supplement comprises a softgel capsule comprising ingredients in amounts sufficient to provide a human with at least about 660.4 mg of docosahexaenoic acid, at least about 401.4 mg of calcium carbonate, at least about 91 mg of ferrous fumarate, at least about 42.9 mg of vitamin C, at least about 37.9 mg of vitamin B6, at least about 30 mg of vitamin E, at least about 0.727 mg of folic acid, at least about 1.45 mg of a folate derivative, at least about 0.99 mg of vitamin D3, and at least about 0.317 mg of D-biotin. The foregoing specific embodiment may further comprise at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, the folate derivative is preferable in the form of (6S)-5-methyltetrahydrofolate glucosamine salt of the form available under the Quatrefolic® trademark, and assigned CAS Number 1181972-37-1.

(400) A supplement for administration to a human, wherein the supplement comprises a softgel capsule comprising ingredients in amounts sufficient to provide a human with at least about 660.4 mg of docosahexaenoic acid, at least about 110 mg of docusate calcium, at least about 401.4 mg of calcium carbonate, at least about 91 mg of ferrous fumarate, at least about 42.9 mg of vitamin C, at least about 37.9 mg of vitamin B6, at least about 30 mg of vitamin E, at least about 0.727 mg of folic acid, at least about 1.45 mg of a folate derivative, at least about 0.99 mg of vitamin D3, and at least about 0.317 mg of D-biotin. The foregoing specific embodiment may further comprise at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, the folate derivative is preferable in the form of (6S)-5-methyltetrahydrofolate glucosamine salt of the form available under the Quatrefolic® trademark, and assigned CAS Number 1181972-37-1.

(401) A supplement for administration to a human, wherein the supplement comprises a softgel capsule comprising ingredients in amounts sufficient to provide a human with at least about 350 mg of docosahexaenoic acid, at least about 157.5 mg of calcium carbonate, at least about 29 mg of ferrous fumarate, at least about 28 mg of vitamin C, at least about 26 mg of vitamin B6, at least about 20.13 mg of vitamin E, at least about 800 mcg of folic acid, at least about 550 mcg of a folate derivative, at least about 20.625 mcg of vitamin D3, and at least about 250 mcg of D-biotin. The foregoing specific embodiment may further comprise at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, the folate derivative is preferable in the form of (6S)-5-methyltetrahydrofolate glucosamine salt of the form available under the Quatrefolic® trademark, and assigned CAS Number 1181972-37-1.

(402) A supplement for administration to a human, wherein the supplement comprises a softgel capsule comprising ingredients in amounts sufficient to provide a human with at least about 350 mg of docosahexaenoic acid, at least about 55 mg of docusate calcium, at least about 157.5 mg of calcium carbonate, at least about 29 mg of ferrous fumarate, at least about 28 mg of vitamin C, at least about 26 mg of vitamin B6, at least about 20.13 mg of vitamin E, at least about 800 mcg of folic acid, at least about 550 mcg of a folate derivative, at least about 20.625 mcg of vitamin D3, and at least about 250 mcg of D-biotin. The foregoing specific embodiment may further comprise at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, the folate derivative is preferable in the form of (6S)-5-methyltetrahydrofolate glucosamine salt of the form available under the Quatrefolic® trademark, and assigned CAS Number 1181972-37-1.

(403) A supplement for administration to a human, wherein the supplement comprises a softgel capsule comprising ingredients in amounts sufficient to provide a human with at least about 660.4 mg of docosahexaenoic acid, at least about 401.4 mg of calcium carbonate, at least about 91 mg of ferrous fumarate, at least about 42.9 mg of vitamin C, at least about 37.9 mg of vitamin B6, at least about 30 mg of vitamin E, at least about 1 mg of folic acid, at least about 750 mcg of a folate derivative, at least about 0.99 mg of vitamin D3, and at least about 0.317 mg of D-biotin. The foregoing specific embodiment may further comprise at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, the folate derivative is preferable in the form of (6S)-5-methyltetrahydrofolate glucosamine salt of the form available under the Quatrefolic® trademark, and assigned CAS Number 1181972-37-1.

(404) A supplement for administration to a human, wherein the supplement comprises a softgel capsule comprising ingredients in amounts sufficient to provide a human with at least about 660.4 mg of docosahexaenoic acid, at least about 110 mg of docusate calcium, at least about 401.4 mg of calcium carbonate, at least about 91 mg of ferrous fumarate, at least about 42.9 mg of vitamin C, at least about 37.9 mg of vitamin B6, at least about 30 mg of vitamin E, at least about 1 mg of folic acid, at least about 750 mcg of a folate derivative, at least about 0.99 mg of vitamin D3, and at least about 0.317 mg of D-biotin. The foregoing specific embodiment may further comprise at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, the folate derivative is preferable in the form of (6S)-5-methyltetrahydrofolate glucosamine salt of the form available under the Quatrefolic® trademark, and assigned CAS Number 1181972-37-1.

(405) A supplement for administration to a human, wherein the supplement comprises a softgel capsule comprising ingredients in amounts sufficient to provide a human with at least about 350 mg of docosahexaenoic acid, at least about 157.5 mg of calcium carbonate, at least about 29 mg of ferrous fumarate, at least about 28 mg of vitamin C, at least about 26 mg of vitamin B6, at least about 20.13 mg of vitamin E, at least about 950 mcg of folic acid, at least about 555.5 mcg of a folate derivative, at least about 20.625 mcg of vitamin D3, and at least about 250 mcg of D-biotin. The foregoing specific embodiment may further comprise at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, the folate derivative is preferable in the form of (6S)-5-methyltetrahydrofolate glucosamine salt of the form available under the Quatrefolic® trademark, and assigned CAS Number 1181972-37-1.

(406) A supplement for administration to a human, wherein the supplement comprises a softgel capsule comprising ingredients in amounts sufficient to provide a human with at least about 350 mg of docosahexaenoic acid, at least about 55 mg of docusate calcium, at least about 157.5 mg of calcium carbonate, at least about 29 mg of ferrous fumarate, at least about 28 mg of vitamin C, at least about 26 mg of vitamin B6, at least about 20.13 mg of vitamin E, at least about 950 mcg of folic acid, at least about 555.5 mcg of a folate derivative, at least about 20.625 mcg of vitamin D3, and at least about 250 mcg of D-biotin. The foregoing specific embodiment may further comprise at least one pharmaceutically acceptable excipient selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks. In the foregoing specific embodiment, docosahexaenoic acid is derived from microalgae—preferably from at least one of Crypthecodinium cohnii, Schizocytrium and Thraustochytrium microalgae, and more preferably from Crypthecodinium cohnii microalgae—and may be provided in the form of a “DHA, 53% in sunflower oil” blend (which may include small amounts of antioxidants (ascorbyl palmitate and alpha-tocopherols)). Moreover, in the foregoing specific embodiment, docusate calcium is preferably in the form of docusate calcium, 50% in corn oil, USP/NF; calcium carbonate is preferably in the form of calcium carbonate, 98.0%, USP; ferrous fumarate is preferably ferrous fumarate, 97.0%, USP; vitamin C is preferably in the form of ascorbic acid, 99%, USP/FCC; vitamin B6 is preferably in the form of pyridoxine HCl, 99%, USP/FCC; vitamin E is preferably in the form of d-alpha-tocopherol, 67.1% in soybean oil; vitamin D3 is preferably in the form of cholecalciferol, 2.5% in corn oil, USP); D-biotin is preferably in the form of D-biotin, 98.5%, USP; folic acid is preferably folic acid, 97%, USP/FCC; and, the folate derivative is preferable in the form of (6S)-5-methyltetrahydrofolate glucosamine salt of the form available under the Quatrefolic® trademark, and assigned CAS Number 1181972-37-1.

### Summary of Manufacturing Process

A process for manufacturing softgel capsules of the various supplements described herein, may be divided into six modules:
(1) Gelatin mass preparation;
(2) Fill material preparation;
(3) Encapsulation (form, fill, seal, and print);
(4) Softgel drying;
(5) Softgel washing, bulk packaging, and final release; and,
(6) Packaging.

Gelatin Mass Preparation. The general process involves the melting of gelatin crystals in a custom gel melting apparatus. Once mixed, the molten gel mass is discharged into specially designed holding tanks which maintain the gel mass at an elevated temperature under constant, slow agitation. Colorants, opacifiers, and flavoring agents are added. Subsequently, the mass is held until needed for encapsulation but must be used within 72 hours after discharging from the melter.

Fill Material Preparation. The fill material is prepared in a series of steps that involve the preparation of several side mixes which are added to the main mix in discrete steps that involve various heating or cooling and mixing cycles. At various points during this process step, the fill material is tested for uniformity.

Encapsulation. Encapsulation is accomplished using a rotary die process. The molten gel mass is cast onto cooling drums and two gelatin ribbons are perpetually formed. These ribbons are cast into a pair of heated, rotating capsule forming dies. As the ribbons are compressed between the dies, the heated fill material is injected between the ribbons to expand the gelatin into the die pockets. As the dies continue rotating, the capsule is completely sealed and separated from the gelatin ribbon. The mixing speeds and operating temperatures are precisely controlled throughout the encapsulation process. Various in-process checks are performed to ensure: content uniformity of fill material, prior to encapsulation; consistent fill weight and seal thickness; and, proper imprinting.

Softgel Drying. Drying is accomplished by loading the capsules into shallow trays which are then stacked and placed into the drying tunnels. The capsules are dried until they meet a specification for hardness at which point they are tested for uniformity and combined into covered deep trays for holding.

Softgel Washing, Bulk Packaging, and Finished Product Testing. Subsequent to drying, the softgels are transferred from the deep trays to roller graders. The softgels are washed with a denatured ethanol/phosal 53 MCT wash solution using a 0.5 1/8 BX spray head with the pressure gauge set at 10 psi. The finished softgels are then inspected for any defects, and if inspection passes, the softgels may be bulk packaged for subsequent re-packaging in smaller bottles or units. In addition, sample finished softgels may be pulled for finished product testing to ensure they conform to finished product acceptance criteria.

While the invention has been described with reference to certain exemplary embodiments thereof, those skilled in the art may make various modifications to the described embodiments of the invention without departing from the scope of the invention. The terms and descriptions used herein are set forth by way of illustration only and not meant as limitations. In particular, although the present invention has been described by way of examples, a variety of compositions and processes would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What is claimed is:

1. A supplement consisting essentially of:
docosahexaenoic acid in an amount of about 350 mg to about 660.4 mg;
docusate calcium in an amount of about 55 mg to about 110 mg;
calcium carbonate in an amount of about 157.5 mg to about 401.4 mg;
ferrous fumarate in an amount of about 29 mg to about 91 mg;
vitamin C in an amount of about 28 mg to about 42.9 mg;
vitamin B6 in an amount of about 26 mg to about 37.9 mg;
vitamin E in an amount of about 20.13 mg to about 30 mg;
folic acid in an amount of about 0.6 mg to about 0.727 mg;
a folate derivative in an amount of about 1.2 mg to about 1.45 mg;
vitamin D3 in an amount of about 20.625 mcg to about 0.99 mg;
D-biotin in an amount of about 250 mcg to about 0.317 mg; and,
at least one pharmaceutically acceptable excipient,
wherein said supplement is formulated as a softgel capsule.

2. The supplement of claim 1, wherein said docosahexaenoic acid is present in an amount of about 350 mg, said docusate calcium is present in an amount of about 55 mg, said calcium carbonate is present in an amount of about 157.5 mg, said ferrous fumarate is present in an amount of about 29 mg, said vitamin C is present in an amount of about 28 mg, said vitamin B6 is present in an amount of about 26 mg, said vitamin E is present in an amount of about 20.13 mg, said folic acid is present in an amount of about 0.6 mg, said folate derivative is present in an amount of about 1.2 mg, said vitamin D3 is present in an amount of about 20.625 mcg, and said D-biotin is present in an amount of about 250 mcg.

3. The supplement of claim 1, wherein said docosahexaenoic acid is present in an amount of about 660.4 mg, said docusate calcium is present in an amount of about 110 mg, said calcium carbonate is present in an amount of about 401.4 mg, said ferrous fumarate is present in an amount of about 91 mg, said vitamin C is present in an amount of about 42.9 mg, said vitamin B6 is present in an amount of about 37.9 mg, said vitamin E is present in an amount of about 30 mg, said folic acid is present in an amount of about 0.727 mg, said folate derivative is present in an amount of about 1.45 mg, said vitamin D3 is present in an amount of about 0.99 mg, and said D-biotin is present in an amount of about 0.317 mg.

4. The supplement of claim 1, wherein said folate derivative is (6S)-5-methyltetrahydrofolate glucosamine salt.

5. The supplement of claim 1, wherein said docosahexaenoic acid is derived from microalgae, and wherein said microalgae is at least one of Crypthecodinium cohnii microalgae, Schizocytrium microalgae, and Thraustochytrium microalgae.

6. The supplement of claim 1, wherein said at least one pharmaceutically acceptable excipient is selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

7. A supplement consisting essentially of:
docosahexaenoic acid in an amount of about 350 mg to about 660.4 mg;
docusate calcium in an amount of about 55 mg to about 110 mg;
calcium carbonate in an amount of about 157.5 mg to about 401.4 mg;
ferrous fumarate in an amount of about 29 mg to about 91 mg;
vitamin C in an amount of about 28 mg to about 42.9 mg;
vitamin B6 in an amount of about 26 mg to about 37.9 mg;
vitamin E in an amount of about 20.13 mg to about 30 mg;
folic acid in an amount of about 800 mcg to about 1 mg;
a folate derivative in an amount of about 550 mcg to about 750 mcg;
vitamin D3 in an amount of about 20.625 mcg to about 0.99 mg;
D-biotin in an amount of about 250 mcg to about 0.317 mg; and,
at least one pharmaceutically acceptable excipient,
wherein said supplement is formulated as a softgel capsule.

8. The supplement of claim 7, wherein said docosahexaenoic acid is present in an amount of about 350 mg, said docusate calcium is present in an amount of about 55 mg, said calcium carbonate is present in an amount of about 157.5 mg, said ferrous fumarate is present in an amount of about 29 mg, said vitamin C is present in an amount of about 28 mg, said vitamin B6 is present in an amount of about 26 mg, said vitamin E is present in an amount of about 20.13 mg, said folic acid is present in an amount of about 800 mcg, said folate derivative is present in an amount of about 550 mcg, said vitamin D3 is present in an amount of about 20.625 mcg, and said D-biotin is present in an amount of about 250 mcg.

9. The supplement of claim 7, wherein said docosahexaenoic acid is present in an amount of about 660.4 mg, said docusate calcium is present in an amount of about 110 mg, said calcium carbonate is present in an amount of about 401.4 mg, said ferrous fumarate is present in an amount of about 91 mg, said vitamin C is present in an amount of about 42.9 mg, said vitamin B6 is present in an amount of about 37.9 mg, said vitamin E is present in an amount of about 30 mg, said folic acid is present in an amount of about 1 mg, said folate derivative is present in an amount of about 750 mcg, said vitamin D3 is present in an amount of about 0.99 mg, and said D-biotin is present in an amount of about 0.317 mg.

10. The supplement of claim 7, wherein said docosahexaenoic acid is present in an amount of about 350 mg, said docusate calcium is present in an amount of about 55 mg, said calcium carbonate is present in an amount of about 157.5 mg, said ferrous fumarate is present in an amount of about 29 mg, said vitamin C is present in an amount of about 28 mg, said vitamin B6 is present in an amount of about 26 mg, said vitamin E is present in an amount of about 20.13 mg, said folic acid is present in an amount of about 555.5 mcg, said folate derivative is present in an amount of about 950 mcg, said vitamin D3 is present in an amount of about 20.625 mcg, and said D-biotin is present in an amount of about 250 mcg.

11. The supplement of claim 7, wherein said folate derivative is (6S)-5-methyltetrahydrofolate glucosamine salt.

12. The supplement of claim 7, wherein said docosahexaenoic acid is derived from microalgae, and wherein said microalgae is at least one of Crypthecodinium cohnii microalgae, Schizocytrium microalgae, and Thraustochytrium microalgae.

13. The supplement of claim 7, wherein said at least one pharmaceutically acceptable excipient is selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

14. A supplement comprising:
docosahexaenoic acid;
calcium carbonate;
ferrous fumarate;
vitamin C;
vitamin B6;
vitamin E;
folic acid;
a folate derivative;
vitamin D3; and,
D-biotin.

15. The supplement of claim 14, wherein said docosahexaenoic acid is present in an amount of about 350 mg to about 660.4 mg, said calcium carbonate is present in an amount of about 157.5 mg to about 401.4 mg, said ferrous fumarate is present in an amount of about 29 mg to about 91 mg, said vitamin C is present in an amount of about 28 mg to about 42.9 mg, said vitamin B6 is present in an amount of about 26 mg to about 37.9 mg, said vitamin E is present in an amount of about 20.13 mg to about 30 mg, said folic acid is present in an amount of about 0.6 mg to about 0.727 mg, said folate derivative is present in an amount of about 1.2 mg to about 1.45 mg, said vitamin D3 is present in an amount of about 20.625 mcg to about 0.99 mg, and said D-biotin is present in an amount of about 250 mcg to about 0.317 mg, and wherein said supplement further comprises docusate calcium in an amount of about 55 mg to about 110 mg.

16. The supplement of claim 14, wherein said docosahexaenoic acid is present in an amount of about 350 mg, said calcium carbonate is present in an amount of about 157.5 mg, said ferrous fumarate is present in an amount of about 29 mg, said vitamin C is present in an amount of about 28 mg, said vitamin B6 is present in an amount of about 26 mg, said vitamin E is present in an amount of about 20.13 mg, said folic acid is present in an amount of about 0.6 mg, said folate derivative is present in an amount of about 1.2 mg, said vitamin D3 is present in an amount of about 20.625 mcg, and said D-biotin is present in an amount of about 250 mcg, and wherein said supplement further comprises docusate calcium in an amount of about 55 mg.

17. The supplement of claim 14, wherein said docosahexaenoic acid is present in an amount of about 660.4 mg, said calcium carbonate is present in an amount of about 401.4 mg, said ferrous fumarate is present in an amount of about 91 mg, said vitamin C is present in an amount of about 42.9 mg, said vitamin B6 is present in an amount of about 37.9 mg, said vitamin E is present in an amount of about 30 mg, said folic acid is present in an amount of about 0.727 mg, said folate derivative is present in an amount of about 1.45 mg, said vitamin D3 is present in an amount of about 0.99 mg, and said D-biotin is present in an amount of about 0.317 mg, and wherein said supplement further comprises docusate calcium in an amount of about 110 mg.

18. The supplement of claim 14, wherein said docosahexaenoic acid is present in an amount of about 350 mg to about 660.4 mg, said calcium carbonate is present in an amount of about 157.5 mg to about 401.4 mg, said ferrous fumarate is present in an amount of about 29 mg to about 91 mg, said vitamin C is present in an amount of about 28 mg to about 42.9 mg, said vitamin B6 is present in an amount of about 26 mg to about 37.9 mg, said vitamin E is present in an amount of about 20.13 mg to about 30 mg, said folic acid is present in an amount of about 800 mcg to about 1 mg, said folate derivative is present in an amount of about 550 mcg to about 750 mcg, said vitamin D3 is present in an amount of about 20.625 mcg to about 0.99 mg, and said D-biotin is present in an amount of about 250 mcg to about 0.317 mg, and wherein said supplement further comprises docusate calcium in an amount of about 55 mg to about 110 mg.

19. The supplement of claim 14, wherein said docosahexaenoic acid is present in an amount of about 350 mg, said calcium carbonate is present in an amount of about 157.5 mg, said ferrous fumarate is present in an amount of about 29 mg, said vitamin C is present in an amount of about 28 mg, said vitamin B6 is present in an amount of about 26 mg, said vitamin E is present in an amount of about 20.13 mg, said folic acid is present in an amount of about 800 mcg, said folate derivative is present in an amount of about 550 mcg, said vitamin D3 is present in an amount of about 20.625 mcg, and said D-biotin is present in an amount of about 250 mcg, and wherein said supplement further comprises docusate calcium in an amount of about 55 mg.

20. The supplement of claim 14, wherein said docosahexaenoic acid is present in an amount of about 660.4 mg, said calcium carbonate is present in an amount of about 401.4 mg, said ferrous fumarate is present in an amount of about 91 mg, said vitamin C is present in an amount of about 42.9 mg, said vitamin B6 is present in an amount of about 37.9 mg, said vitamin E is present in an amount of about 30 mg, said folic acid is present in an amount of about 1 mg, said folate derivative is present in an amount of about 750 mcg, said vitamin D3 is present in an amount of about 0.99 mg, and said D-biotin is present in an amount of about 0.317 mg, and wherein said supplement further comprises docusate calcium in an amount of about 110 mg.

21. The supplement of claim 14, wherein said docosahexaenoic acid is present in an amount of about 350 mg, said calcium carbonate is present in an amount of about 157.5 mg, said ferrous fumarate is present in an amount of about 29 mg, said vitamin C is present in an amount of about 28 mg, said vitamin B6 is present in an amount of about 26 mg, said vitamin E is present in an amount of about 20.13 mg, said folic acid is present in an amount of about 950 mcg, said folate derivative is present in an amount of about 555.5 mcg, said vitamin D3 is present in an amount of about 20.625 mcg, and said D-biotin is present in an amount of about 250 mcg, and wherein said supplement further comprises docusate calcium in an amount of about 55 mg.

22. The supplement of claim 14, wherein said folate derivative is (6S)-5-methyltetrahydrofolate glucosamine salt.

23. The supplement of claim 14, wherein said docosahexaenoic acid is derived from microalgae, and wherein said microalgae is at least one of Crypthecodinium cohnii microalgae, Schizocytrium microalgae, and Thraustochytrium microalgae.

24. The supplement of claim 14, wherein said at least one pharmaceutically acceptable excipient is selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

25. The supplement of claim 14, further comprising at least one ingredient select from the group consisting of vitamin A, vitamin C, vitamin D, vitamin E, vitamin B1, vitamin B2, vitamin B3, vitamin B5, vitamin B6, vitamin B12, choline, vitamin D3, D-biotin, iodine, iron, zinc, copper, calcium, magnesium, an omega-3 fatty acid, and any pharmaceutically acceptable forms thereof.

26. The supplement of claim 14, wherein said supplement is formulated as a swallowable, single dosage form, and wherein said dosage form is a softgel capsule.

27. A supplement comprising:
docosahexaenoic acid in an amount of at least about 350 mg;
calcium in an amount of at least about 160 mg;
ferrous fumarate in an amount of at least about 29 mg;
vitamin C in an amount of at least about 28 mg;
vitamin B6 in an amount of at least about 26 mg;
vitamin E in an amount of at least about 20.13 mg;
folic acid in an amount of at least about 0.6 mg;
(6S)-5-methyltetrahydrofolate glucosamine salt in an amount of at least about 1.2 mg;
vitamin D3 in an amount of at least about 20.625 mcg; and,
D-biotin in an amount of at least about 250 mcg.

28. The supplement of claim 27, further comprising docusate calcium in an amount of at least about 55 mg.

29. The supplement of claim 28, wherein at least about 157.5 mg of said at least about 160 mg of calcium is in the form of calcium carbonate and, optionally, wherein at least about 2 mg of said at least about 160 mg of calcium is provided by said at least about 55 mg of docusate calcium.

30. The supplement of claim 27, wherein said docosahexaenoic acid is derived from microalgae, and wherein said microalgae is at least one of Crypthecodinium cohnii microalgae, Schizocytrium microalgae, and Thraustochytrium microalgae.

31. The supplement of claim 27, wherein said at least one pharmaceutically acceptable excipient is selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

32. The supplement of claim 27, further comprising at least one ingredient select from the group consisting of vitamin A, vitamin C, vitamin D, vitamin E, vitamin B1, vitamin B2, vitamin B3, vitamin B5, vitamin B6, vitamin B12, choline, vitamin D3, D-biotin, iodine, iron, zinc, copper, calcium, magnesium, an omega-3 fatty acid, and any pharmaceutically acceptable forms thereof.

33. The supplement of claim 27, wherein said supplement is formulated as a swallowable, single dosage form, and wherein said dosage form is a softgel capsule.

34. A supplement for administration to a human, said supplement comprising:
a softgel capsule comprising ingredients in amounts sufficient to provide a human with at least about 350 mg of docosahexaenoic acid, at least about 157.5 mg of calcium carbonate, at least about 29 mg of ferrous fumarate, at least about 28 mg of vitamin C, at least about 26 mg of vitamin B6, at least about 20.13 mg of vitamin E, at least about 0.6 mg of folic acid, at least about 1.2 mg of a folate derivative, at least about 20.625 mcg of vitamin D3, and at least about 250 mcg of D-biotin.

35. The supplement of claim 34, further comprising at least about 55 mg of docusate calcium.

36. The supplement of claim 34, wherein said folate derivative is (6S)-5-methyltetrahydrofolate glucosamine salt.

37. The supplement of claim 34, wherein said docosahexaenoic acid is derived from microalgae, and wherein said microalgae is at least one of Crypthecodinium cohnii microalgae, Schizocytrium microalgae, and Thraustochytrium microalgae.

38. The supplement of claim 34, further comprising at least one pharmaceutically acceptable excipient is selected from the group consisting of carriers, binders, fillers, diluents, viscosity agents, thickening agents, colorants, emulsifiers, lubricants, glidants, disintegrants, water, oils, alcohols, preservatives, flavorants, sweetening agents, sugars, coatings and pharmaceutical inks.

39. The supplement of claim 34, further comprising at least one ingredient select from the group consisting of vitamin A, vitamin C, vitamin D, vitamin E, vitamin B1, vitamin B2, vitamin B3, vitamin B5, vitamin B6, vitamin B12, choline, vitamin D3, D-biotin, iodine, iron, zinc, copper, calcium, magnesium, an omega-3 fatty acid, and any pharmaceutically acceptable forms thereof.
